(19) United States
(12) Patent Application Publication
(10) Pub. No.: US 2009/0155272 A1
(43) Pub. Date: Jun. 18, 2009

(54) Targeted pharmaceuticals and ligands

(76) Inventors: John L. Muschler, Albany, CA (US); Dmitri B. Kirpotin, San Francisco, CA (US); Daryl C. Drummond, Pacifica, CA (US)

Correspondence Address: BUCHANAN, INGERSOLL & ROONEY LLP, P.O. BOX 1404, ALEXANDRIA, VA, 22313-1404, US

(21) Appl. No.: 11/991,102

(22) PCT Filed: Sep. 29, 2006
(86) PCT No.: PCT/US2006/038249

§ 371 Date: Feb. 27, 2008

Related U.S. Application Data
(60) Provisional application No. 60/722,925, filed on Sep. 30, 2005.

(51) Int. Cl. A61K 39/395; A61K 9/127; A61K 31/7088; A61K 38/02; C40B 30/06; A61K 39/00

(52) U.S. Cl. 424/135.1; 424/450; 514/44; 514/2; 424/130.1; 506/10; 424/277.1

(57) ABSTRACT

The disclosure pertains to pharmaceuticals, more specifically, to targeted diagnostic and therapeutic formulations and ligands thereof. Such methods and composition comprise antigens that are post-translationally modified compared to antigens found on a normal cell phenotype. Also provided are ligands that bind to such post-translationally modified antigens.

### CROSS REFERENCE TO RELATED APPLICATIONS

This application claims priority to provisional application 60/722,925, filed Sep. 30, 2005, the disclosure of which is incorporated herein by reference.

### FIELD OF THE INVENTION

The invention pertains to pharmaceuticals, more specifically, to targeted diagnostic and therapeutic formulations and ligands thereof.

### BACKGROUND

Conventional cancer chemotherapeutic agents cannot distinguish between normal cells and tumor cells and hence damage and kill normal proliferating tissues. One approach to reduce this toxic side effect is to specifically target the chemotherapeutic agent to the tumor. This is the rationale behind the development of immunotoxins, chimeric molecules composed of an antibody either chemically conjugated or fused to a toxin that binds specifically to antigens on the surface of a tumor cell thereby killing or inhibiting the growth of the cell. The majority of immunotoxins prepared to date, have been made using murine monoclonal antibodies (MAbs) that exhibit specificity for tumor cells. Immunotoxins made from MAbs demonstrate relatively selective killing of tumor cells in vitro and tumor regression in animal models.

Despite these promising results, the use of immunotoxins in humans has been limited by toxicity, immunogenicity and a failure to identify highly specific tumor antigens. Nonspecific toxicity results from the failure of the monoclonal antibody to bind specifically and with high affinity to tumor cells. As a result, nonspecific cell killing occurs.

In recent years, molecular-targeted interventions and nanocarrier-based drug delivery are gaining an increasing acceptance into standard arsenal of clinical oncology. Current efforts have been focused on tumor recognition markers, such as surface antigens and receptors, whose specificity arises from translationally defined events, such as gene modification and alternative splicing. The usefulness of these markers is often limited by their insufficient cellular abundance or limited occurrence within a patient population, or by a limited gene copy number and selection for low expressing cellular types during the progress of disease. Therefore, there is an unmet need, addressed by this invention, for better molecular therapeutic targets for therapy, diagnosis or prevention of diseases, in particular, those characterized by a malignant neoplastic process.

### SUMMARY

This invention addresses the need for better targeted anti-cancer therapeutics. The inventive approach employs cancer-specific post-translational modifications that occur during cancer pathogenesis as markers for nanocarrier-based ligand-directed targeted drug delivery.

The invention provides targeting ligands that bind to post-translationally modified antigens (PTMA) on the surface of target cells, for example, cancer cells. The targeting ligands may be comprised with a therapeutic, diagnostic or prophylactic pharmaceutical composition or article, alone or in combination with a drug or detectable marker. In one embodiment, the invention provides such ligands in the form of Fv, or single chain Fv (scFv) antibody fragments, combined with cytotoxic drug-loaded nanosized drug carriers, such as liposomes. In another embodiment, the PTMA-binding ligands, and/or ligand-linked nanosized carriers are internalized into the diseased cells. In one preferred embodiment, the PTMA is a post-translationally modified variant of dystroglycan (DG).

The invention also provides ligand library selection methods, such as, for example, phage display library selection, and antibody-liposome uptake screening methods on live cells to identify the ligands that bind to PTMA. In one embodiment, the library selection method includes selection for the ligand library members that bind and are internalized by cells that display post-translationally modified entities.

The invention also provides immunological tools for detections of diagnostic and prognostic biomarkers of cancers (e.g., breast cancer), as well as valuable research tools to assay post-translational modifications of cancer cell proteins both in vitro and or in vivo, are generated.

The invention provides one method to screen and identify a ligand that interacts with a post-translationally modified antigen (PTMA) comprising: (a) contacting a ligand library having a plurality of members with cells that display the PTMA, and (b) separating the cells from the library members that do not associate with the cells. The invention also provides another method of identifying a ligand that interacts with a post-translationally modified antigen (PTMA) comprising: (a) contacting a ligand library (e.g., a scFv phage display library) having a plurality of members with cells of a first cell line that displays a precursor antigen; (b) separating the ligand library members that do not associate with the cells of the first cell line; and (c) contacting the ligand display library members separated in step (b) with cells of the second cell line, wherein the second cell line is obtained by a process comprising inducing the first cell line to modify the precursor antigen into the post-translationally modified antigen. In one preferred instance, the method entails exposing a library (e.g., a phage display library) of anti-PTMA-ligands (e.g., single chain antibodies) to a first cell line having a normal phenotype; allowing the first cell line and library to interact; removing bound cells from the library to provide a library enriched with members comprising PTMA-specific ligands; contacting the enriched library with a second cell line expressing a post-translationally modified phenotype and identifying enriched library members the bind to the second cell line. In one aspect, the first cell line is modified by contacting the cell line with an agent that causes modification of the genotype or phenotype of the cell line to generate a second cell line. In a further aspect, the library is enriched with, and selected for, ligands, e.g. antibodies, that mediated internalization subsequent to cell-surface binding. In a further aspect, the PTMA-reactive ligands, such as of the scFvs selected as above with stability and the ability to mediate internalization are assayed for particular applications in a routine manner.

The invention further provides chimeric anti-PTMA ligands comprising the anti-PTMA-ligand and a biologically active agent. In one embodiment, the anti-PTMA-ligand is identified by the methods in the preceding paragraph. In another embodiment, anti-PTMA scFvs are conjugated to an amphiphilic linker molecule, such as, for example, maleimide-PEG-distearoylphosphatidylethanolamine (Mal-PEG-DSPE), and the resulting micelle-forming conjugate is linked to a diagnostic, e.g., fluorescent, probe- or drug-loaded liposomes using micellar insertion method.

Anti-PTMA immunoliposomes are also provided by the invention. Such anti-PTMA-immunoliposomes are routinely characterized in vivo for pharmacokinetics, biodistribution, toxicity, and anti-tumor efficacy. In one aspect, the antitumor efficacy is determined in a number of human breast cancer xenograft models with various expression levels of the targeted epitope using liposomes and anti-PTMA immunoliposomes loaded with anticancer drugs, such as anthracyclines (e.g., doxorubicin), vinca alkaloids (e.g., vinorelbine), or camptothecins (e.g., topotecan or irinotecan).

Compositions comprising a PTMA-antigen is also provided. Such compositions can include adjuvants to elicit an immune response. Such compositions can prove useful as vaccines.

Also provided are antigen presenting cells recombinantly modified to express a PTMA-antigen identified by the invention. In one aspect, the PTMA-antigen is a hypoglycosylated DG moiety.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

FIG. 1A-E illustrate a variant of phage display technique for selecting target-specific internalizing peptide ligands, such as scFv antibodies, from a phage display library using living cells under in vitro conditions. A) Deplete the number of non-specifically binding/internalizing members of a phage display library by incubation with control cell line that lacks sufficient number of target epitopes; B) Incubate the depleted library with the target cells expressing the target epitope in sufficient number under the conditions (e.g., 37° C.) that allow the epitope/phage complex to internalize; C) Remove all extracellular phages, including externally bound ones, by washing under epitope-antibody dissociative conditions (e.g., low pH buffer and/or trypsinization); D) Lyse the cells and recover the internalized phage particles; E) Re-infect the recovered phage into bacterial host and propagate the phage. F) Repeat selection process as needed to obtain a number of highly specific internalizing clones.

FIG. 2A-G shows internalization of anti-HER2 immunoliposomes containing gold particles by HER2-overexpressing human breast carcinoma (BT474) cells in vivo (A-C). Nontargeted liposomes are shown to accumulate in perivascular regions or inside tumor residing macrophages in BT474 xenografts (D,E). Likewise, human breast carcinoma xenografts containing only low quantities of HER2 (i.e. 10,000 copies/cell) were shown to result in a similar distribution of anti-HER2 immunoliposomes and nontargeted liposomes (F,G).

FIG. 3A-B depicts one method for chemical conjugation (A) and liposome modification (B) technologies to produce a ligand-linked nanocarrier. scFv antibody fragments containing a single engineered cysteine in their c-terminus are initially conjugated to maleimide-functionalized poly(ethylene glycol)-distearoylphosphatidylethanol-amine lipid anchors (A). The scFv conjugates are subsequently incubated with preformed and already drug-loaded liposomes to transform an inert liposomal therapeutic into a molecularly targeted immunoliposomal therapeutic (B).

FIG. 4 shows antitumor efficacy of HER2-targeted (F5-ILs-Dox) and nontargeted (Ls-Dox) liposomal doxorubicin formulations with various amounts of PEG-DSPE lipopolymer (as mol. % of total phospholipid) in human HER2-overexpressing BT474 xenograft tumors. Mice were treated with three weekly i.v. injections (days 13, 20, and 27) equivalent to 5 mg/kg of doxorubicin) starting when the tumor reached an average size of 250 mm³ for a total of three injections. Tumors were measured by caliper twice weekly and the tumor volumes were calculated as ab²/2, where a and b are the tumor length and width, respectively.

FIG. 5 depicts a hypothetical model of the processing events modulating α-DG function. In normal cells and noninvasive carcinoma cells, the glycosylation of α-DG takes place in the Golgi, creating a functional laminin-binding epitope. In addition, furin cleaves α-DG to release the CN-DG molecule. The mature α-DG/β-DG dimer assembles with unknown DG-associated molecules (oval molecule) in the membrane. At the cell surface, MP-dependent cleavage of a DG-associated protein(s) leads to dissociation of the dystroglycan complex, resulting in the shedding of a-DG from the cell surface. This in turn renders the 43-kDa β-DG molecule susceptible to MP-dependent cleavage to form a 31-kDa variant that is internalized and degraded through the proteasome pathway. These same events occur in invasive carcinoma cells, except that initial glycosylation steps are defective, resulting in a completely nonfunctional α-DG (i.e., one that is unable to bind to basement membrane components).

FIG. 6A-B depicts a modification of dystroglycan by metalloprotease activity. (A) Structure of α-DG and β-DG. α-DG associates non-covalently with β-DG, which spans the plasma membrane. O-linked glycosylation is shown as a chain of circles of varying length, whereas N-linked glycosylation is shown as branches. (B) Immunoblot analysis of α-DG and β-DG levels in cell lines cultured in the presence or absence of the MP inhibitor BB-2516. Cells were cultured in serum-free medium for two days with or without BB-2516 and then total cellular proteins were extracted and subjected to immunoblot analysis of α-DG and β-DG.

FIG. 7A-B showed altered processing of α-DG, and loss of DG function in invasive breast cancer cell lines (A) Immunoprecipitation of the DG complex form EpH4, MDA-MB-231 and MDA-MB-468 cells revealed bands corresponding to α-DG at 150 kDa in normal cells (EpH4) and ˜100 kDa in carcinoma cells. The bands were shifted upward in all cells following treatment with the furin inhibitor, CMK, revealing a common N-terminus. (B) Laminin binding. Proteins from FIG. 7A were assayed for laminin binding by laminin-overlay. Laminin binding was detected in normal cells, but not in carcinoma cells.

FIG. 8A-C shows expression of wild type (WT) and mutant DG cDNA in DG−/− cells, and assays of laminin assembly. Cells completely lacking DG expression were infected with a retroviral vector (V), viruses encoding the wild type DG cDNA (WT) or mutant DG cDNAs encoding an internal cytoplasmic domain deletion (D1), mutated α/β cleavage site (MC), and mutated transferase recognition site (HG). (A) immunoblots for α-DG and β-DG shows the absence of DG in the “V” population, and expression of WT and mutant DG proteins in the others. (B) Cells exposed to fluorecein-lableled laminin-1 were assessed for laminin assembly at the cell surface by fluorescence microscopy. Laminin binding and assembly is completely absent in cells lacking DG, and evident in all cells expressing the WT or mutant DG proteins (although greatly reduced with the HG mutant). (C) Quantification of laminin assembly at the cell surface reveals the relative laminin binding in each population.

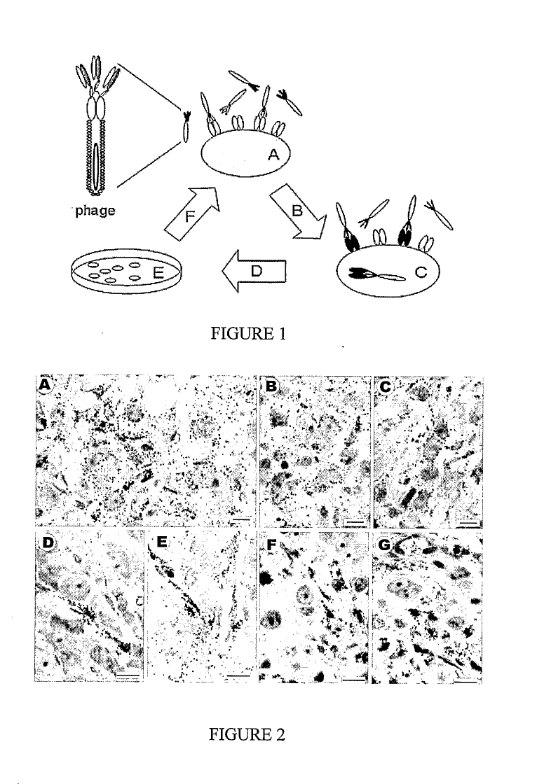

### DETAILED DESCRIPTION

Unless defined otherwise, all technical and scientific terms used herein have the same meaning as is commonly understood by one of skill in the art to which this invention belongs. All publications and patents referred to herein are incorporated by reference.

As used herein and in the appended claims, the singular forms “a,” “and,” and “the” include plural referents unless the context clearly dictates otherwise. Thus, for example, reference to “an antibody” includes a plurality of such antibodies and reference to “the antigen” includes reference to one or more antigens known to those skilled in the art, and so forth.

Diseased cells in the body often display specific molecular markers. These markers may be, for example, proteins, polysaccharides, lipids, their derivatives and combinations. Knowledge of such markers and of ligands that specifically bind to such markers has important practical application as it helps to diagnose the disease and direct treatments such as cytotoxins, drugs, genes, or radioisotopes specifically toward diseased (target) cells while sparing normal cells.

Some markers are expressed on the surface of cells as they are produced by the cell, for example, as encoded by the cellular DNA, and further modified during intracellular processing on the level of DNA and/or mRNA, i.e. on the translation level. Others, however, are expressed as normal cell surface components and are later, i.e., post-translationally, modified into a disease-specific form, for example under the influence of an enzyme or other factor present on or around the surface of the diseased cell. These markers are referred to as phenotypically derived antigens or markers. The molecule that undergoes such modification to yield a phenotypically derived marker is referred to as a precursor antigen. The precursor antigen may consist of one or more molecules, and undergo post-translational modification while still intracellularly, or when already on the cell surface.

Modifications are evident in polypeptides and glycoproteins present at the surface of diseased cells. These modifications include altered glycosylation, protein cleavage, and possibly altered protein folding (e.g., prions, or as the results of physiological conditions like hypoxia and pH). These modifications produce novel epitopes and/or newly exposed protein components that can be recognized and bound by other molecular agents (e.g., antibodies). Examples of such modifications are provided herein as they relate to the cell-surface protein “dystroglycan” (DG) in carcinoma cells. The invention provides a method for selecting disease-specific binding and/or internalizing ligands to post-translationally modified proteins and employing ligands specific for these modifications for the purpose of identifying diseased cells in tissue biopsies and targeting diseased cells for selective treatment.

The diversity of antigens at the cancer cell surface is extremely rich when taking into account not just the changes in gene expression, but also the changes in post-translational protein modifications that occur at the cell surface. Variation in post-translational modifications arises from changes in the internal processing of proteins (e.g., altered glycosylation), and they arise from modifications at the cell surface (e.g., protease cleavage events). The variability in protein post-translational modifications in cancer cells result from the same factors that produce an altered cell behavior, including genetic modifications (gene amplification, deletion or mutation), mis-regulated signaling pathways, and altered signaling and biochemical milieu created from changes in the cellular microenvironment that accompany the progression of cancers. The unique combination of these factors at the sight of tumor growth can, in theory, produce an exponential increase in the diversity of antigens present at the cancer cell surface. Particular modifications may be greatly enhanced in cancer cells relative to normal cells, or they may be entirely unique to cancer cells.

While some works have attempted to exploit post-translational modification in the past, this molecular diversity has been largely ignored in previous screens for tumor-specific antigens. Some antibodies have been generated against protease-generated “neoepitopes” (newly created or newly exposed antibody-binding epitopes) and have been used to assay cleavage events in secreted molecules related to disease states, including Alzheimer's and cancers; however, no antibodies have yet been created that bind to the neoepitopes created by proteases acting on cell-surface molecules. Similarly, few antibodies exist that distinguish cell-surface molecular isoforms created by altered glycosylation, and none of these are currently being employed for diagnostic or therapeutic approaches. Moreover, many cell-surface molecules, including, as herein described, those modified by post-translational modifications, are internalized upon ligand binding, indicating that many post-translational protein modifications represent valuable antigens for antibody-directed targeting of liposomes.

The invention demonstrates that a large, diverse and untapped pool of cancer-specific antigens exists among the post-translational modifications of cell surface proteins in cancer cells, and that ligands, for example, antibodies or antibody fragments, that bind to these modified proteins, mediate internalization of bound chemotherapeutic agents, thereby providing a much more diverse and useful arsenal of antibody-therapeutic constructs than currently available.

One advantage of the invention is the creation of new and more efficient targets for the targeting of therapies or diagnostics to diseased cells. The selection of targeting ligands using various approaches that take advantage of post-translational modifications that occur during pathogenesis, and the subsequent targeting of these antigens to deliver a therapeutic agent or diagnostic agent is provided. The therapeutic agent may include the targeting molecule itself, in the case where receptor binding results in therapeutic activity, or may be a small molecule, biological, or macromolecularly-delivered therapeutic linked covalently or noncovalently to the targeting ligand.

The invention demonstrates that certain post-translational modifications, such as proteolysis and altered glycosylation, of otherwise benign cell surface proteins create structural and functional specificity characteristics for cancer cells, and therefore hold promise as widespread, abundant and specific targets for cancer cell-directed therapy.

An “antigen”, as used herein, refers to any molecular moiety found in, or on, a living cell, including, but not limited to, those molecular moieties that specifically bind to antibodies. A “target PTMA, or “PTMA-antigen” refers to an antigen that is post-translationally modified compared to a normal or control sample.

A “targeting ligand” or a “ligand” refers to a molecular moiety that binds to an antigen. “Anti-PTMA ligand” is a ligand that binds to a PTMA-antigen. Examples of a targeting ligand or anti-PTMA ligand include, but are not limited to, an antibody, an antibody fragment, a polypeptide moiety, a nucleic acid, an aptamer, a small molecule and a peptidomimetic.

As used herein, an “antibody” refers to a polypeptide comprising one or more domains substantially encoded by immunoglobulin genes or fragments of immunoglobulin genes. The recognized immunoglobulin genes include the kappa, lambda, alpha, gamma, delta, epsilon and mu constant region genes, as well as a myriad of immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains, respectively.

Antibodies exist as intact immunoglobulins or as a number of well characterized fragments produced by digestion with various peptidases. Thus, for example, pepsin digests an antibody below the disulfide linkages in the hinge region to produce F(ab)′2, a dimer of Fab which itself is a light chain joined to VH-CH by a disulfide bond. The F(ab)′2 may be reduced under mild conditions to break the disulfide linkage in the hinge region thereby converting the (Fab′)2 dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing molecular biology techniques. Thus, the term antibody, as used herein also includes antibody fragments either produced by the modification of whole antibodies or synthesized de novo using recombinant DNA methodologies.

An “antigen-binding site” or “binding portion” refers to the part of an immunoglobulin molecule that participates in antigen binding. The antigen binding site is formed by amino acid residues of the N-terminal variable (“V”) regions of the heavy (“H”) and light (“L”) chains. Three highly divergent stretches within the “V” regions of the heavy and light chains are referred to as “hypervariable regions” which are interposed between more conserved flanking stretches known as “framework regions” or “FRs”. Thus, the term “FR” refers to a domain comprising an amino acid sequences which are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen binding “surface”. This surface mediates recognition and binding of the target antigen. The three hypervariable regions of each of the heavy and light chains are referred to as “complementarity determining regions” or “CDRs”.

As used herein, the terms “immunological binding” and “immunological binding properties” refer to the non-covalent interactions of the type that occur between an immunoglobulin molecule and an antigen for which the immunoglobulin is specific. The strength or affinity of immunological binding interactions can be expressed in terms of the dissociation constant (Kd) of the interaction, wherein a smaller Kd represents a greater affinity. Immunological binding properties of selected polypeptides can be quantified using methods known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” and the “off rate constant” can be determined by calculation of the concentrations and the actual rates of association and dissociation. The ratio of off/on enables cancellation of all parameters not related to affinity and is thus equal to the dissociation constant Kd.

A single chain Fv (“sFv” or “scFv”) polypeptide is a covalently linked VH::VL heterodimer which may be expressed from a polynucleotide including VH- and VL-encoding sequences either joined directly or joined by a peptide-encoding linker. A number of structures for converting the naturally aggregated—but chemically separated light and heavy polypeptide chains from an antibody “V” region into an sFv molecule which will fold into a three dimensional structure substantially similar to the structure of an antigen-binding site.

The phrase “specifically binds” or “specifically immunoreactive with”, when referring to an antibody, or other ligand, refers to a binding reaction which is determinative of the presence of an antigen in the presence of a heterogeneous population of molecules and other biologics. Thus, under designated immunoassay conditions, the specified antibody binds to a particular antigen (e.g., a specific cell surface polypeptide) and does not bind in a significant amount to other antigenic molecules present in the sample. Specific binding to an antigen under such conditions may require an antibody that is selected for its specificity for a particular antigen. A variety of immunoassay formats may be used to select antibodies specifically immunoreactive with a particular antigen. For example, solid-phase ELISA immunoassays are routinely used to select monoclonal antibodies specifically immunoreactive with an antigen.

To create higher affinity antibodies, mutant sFv gene repertories, based on the sequence of a binding sFv, are created and expressed on the surface of phage. Higher affinity sFvs are selected on antigen as described herein. One approach for creating mutant sFv gene repertoires has been to replace either the VH or VL gene from a binding sFv with a repertoire of nonimmune VH or VL genes (chain shuffling). Such gene repertoires contain numerous variable genes derived from the same germline gene as the binding sFv, but with point mutations. Using light chain shuffling and phage display, the binding avidities of a human sFv antibody fragment can be dramatically increased.

As defined above, an “antigen” also includes, without limitation, a molecule comprising one or more epitopes that generates an immune response. In one aspect, of the invention an antigen used in the compositions and methods of the invention comprises a cell surface polypeptide post-translationally modified (a PTMA-antigen) differently in a cell comprising a cell proliferative disorder than a normal/standard cell. In one aspect, a post-translational modification comprises glycosylation (e.g., hyper- or hypo-glycosylation) of a cell surface polypeptide. An PTMA-antigen may be formulated into a composition either alone or in combination with an adjuvant to provide a vaccine useful for producing an immune response to the PTMA-antigen. Such an immune response may provide protective immunity (e.g., a cancer vaccine).

A biological active agent refers to an agent capable of eliciting a biological change in a cell (e.g., promoting cell death, apoptosis, decrease in cell proliferative capacity, decrease in mitogenic activity, decrease in migration, inhibiting vascularization, inhibiting angiogenesis and the like). For example, a biological active agent can be selected from the group consisting of a cytotoxin (e.g., PE, DT, Ricin A, and the like), a label, a radionuclide, a liposome, a ligand, a nanoparticle, a pharmacological agent (e.g., a drug) or a vehicle containing a pharmacological agent and the like. An encapsulation system, such as a liposome or micelle that contains a biological active agent, such as a drug, a nucleic acid (e.g. an antisense nucleic acid), or another therapeutic moiety can be used to shield the agent from direct exposure to the circulatory system. Means of preparing liposomes attached to antibodies are known to those of skill in the art. An antibody, for example, may be chemically conjugated to a biological active agent. Thus, an antibody may be conjugated to a drug such as vinblastine, vindesine, melphalan, N-Acetylmelphalan, methotrexate, aminopterin, doxorubicin, daunorubicin, genistein (a tyrosine kinase inhibitor), an antisense molecule, and other pharmacological agents known to those of skill in the art, thereby specifically targeting the biological active agent to tumor cells comprising a postranslationally modified cell surface polypeptide.

Detectable labels may also be linked to an anti-PTMA-ligand (e.g., antibody). Such labels may be detectable by spectroscopic, photochemical, biochemical, immunochemical, electrical, optical or chemical means. Useful labels include magnetic beads, fluorescent dyes (e.g., fluorescein isothiocyanate, texas red, rhodamine, green fluorescent protein, and the like), radiolabels (e.g., ³H, ¹²⁵I, ³⁵S, ¹⁴C, or ³²P), nanoparticles, enzymes (e.g., horse radish peroxidase, alkaline phosphatase and others commonly used in an ELISA), and colorimetric labels such as colloidal gold or colored glass or plastic (e.g. polystyrene, polypropylene, latex, and the like) beads. In vivo detection labels may also be used to localize cells comprising post-translationally modified polypeptides indicative of a cell proliferative disorder. Such detection involves administering to an organism a label detectable in vivo. Such labels are known to those of skill in the art and include, but are not limited to, electron dense labels such as gold or barium which may be detected by X-ray or CAT scan, various radioactive labels that may be detected using scintillography, and various magnetic and paramagnetic materials that may be detected using positron emission tomography (PET) and magnetic resonance imaging (MRI).

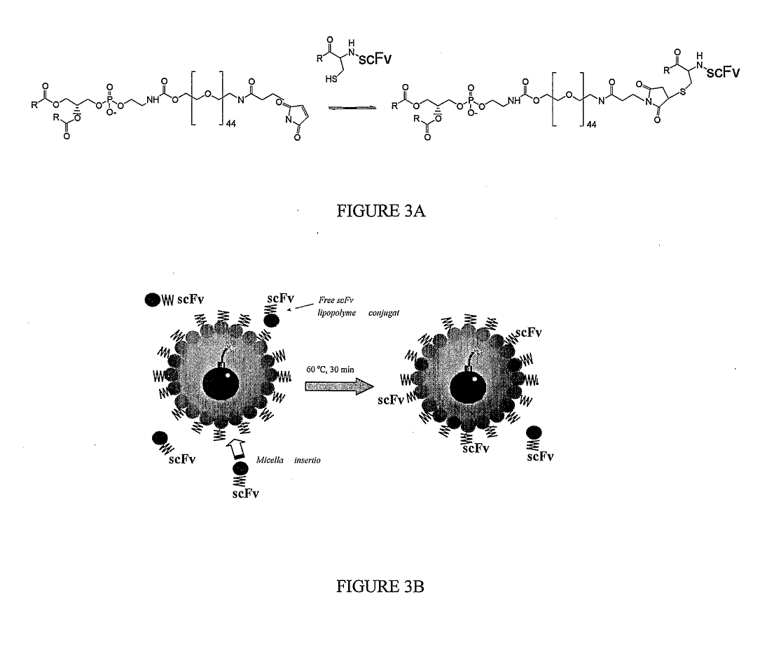

Any method known in the art can be employed to link an anti-PTMA ligand (e.g., an antibody) to a biologically active agent or diagnostic agent, such as a cytotoxin molecule, a pharmaceutical compound, a drug carrier, a nanoparticle, a detectable carrier, or a liposome. One particularly method of conjugating an anti-PTMA ligand, such as an antibody, a fragment thereof, or a scFv to a liposome is disclosed in U.S. Pat. No. 6,210,707; U.S. Pat. No. 6,528,087; U.S. Pat. No. 6,803,053, whose disclosures are incorporated herein by reference. This method sometimes termed as “micellar insertion”, involves incubation of a scFv-polymer-lipid conjugate with preformed liposomes containing encapsulated drug. The conjugates transfer out of the micelles and into the outer monolayer of the liposomal membrane, transforming the liposomes into molecularly targeted liposomal therapeutics (FIG. 3B). This technology is invaluable in the scale-up/translation of the final construct due to the ease and reproducibility of manufacturing. The conjugation strategy also allows for conjugation to a single well engineered cysteine in the c-terminus of the scFv fragment thus allowing for correct orientation of the scFv away from the liposome and allowing for optimal binding to its target.

The invention provides methods and compositions useful for targeting biological active agents to cancer cells. The invention utilizes the post-translational differences in cell surface proteins between normal and cancer cells as a marker and target for cancer cell diagnostics and therapeutics. For example, the invention identifies post-translationally created molecular signatures of breast cancer (post-translationally modified antigens, PTMA). These PTMAs can be employed for targeted therapeutic drug delivery specific to malignant cells, with surprising and unexpected advantage over current targeted anticancer therapeutics. Accordingly, this invention encompasses a PTMA-directed diagnostic and/or therapeutic composition. In one aspect, the PTMA-directed agent is a drug nanocarrier.

For example, in one aspect, the invention uses experience in the drug delivery art with the phage display-generated single chain Fv (scFv) as targeting ligands for cytotoxic nanosized liposome carriers, the invention utilizes a process of identifying targeting agents comprising: selecting human scFv antibodies specific to post-translationally modified antigens characteristic to cancer cells (e.g., breast cancer) using live cell phage display library panning and selection for cell-internalized phages; and creating immunoliposomal carriers loaded with cytotoxic drugs conjugated to selected scFvs as targeting ligands. The specificity, drug delivery properties, and antitumor efficacy of these PTMA-targeted immunoliposomal carries in vitro and in vivo is advantageous over the existing targeting compositions. The PTMA-targeted drug delivery creates new possibilities for better treatment of breast cancer, other cancers, other cell proliferative diseases, and generally, other diseases as well, and, further, the invention addresses the unmet need of expanding the eligibility for targeted drug delivery into larger population of breast cancer subjects. The invention also encompasses new immunological reagents for the diagnosis and molecular characterization of breast cancer.

The relevance of post-translational modifications in diseased cells is illustrated here by the modifications of dystroglycan (DG) in carcinoma cells. In general, PTMA that form through post-translational events characteristic for the distinctive phenotype of the cell, for example, a pathological state or transformation, are herein referred to as “phenotypically derived”, while moieties that via such post-translational events give rise to such PTMA, are herein referred to as “precursors”. Dystroglycan forms the core of a transmembrane protein complex that mediates cell-extracellular matrix (ECM) interactions in muscle, neuronal and epithelial cells. There are multiple post-translational events that modify the composition and function of this receptor complex at the cell surface. These events include destabilization of the complex, release of the alpha-DG subunit from the cell surface, and metalloprotease-mediated cleavage of the beta-DG subunit. The disclosure provides evidence to indicate that these post-translational modifications often occur in the course of cell growth and tissue remodeling, but are suppressed during normal tissue homeostasis. Conditions that trigger modification of the complex include synergy between TGF-beta signaling, oncogene activity (Ras), and loss of normal cell-ECM interactions. These conditions are all evident at the sight of cancer growth. Therefore, modifications of the DG protein complex can be unique to, or highly elevated in cancers cells. In this example, a precursor, i.e., normal tissue DG, through post-translational processing characteristic for a distinctive (malignant) phenotype of the cell, gives rise to a dissociated and partially cleaved DG, which is, therefore, said to be phenotypically derived PTMA.

In addition to the regulation of the DG complex described above, the invention also provides an aberrant form of alpha-DG displayed on the surface of many carcinoma cells. This form is particularly evident in advanced (invasive) cancer cell models. The origin of this defect is hypo-glycosylation of the alpha-DG subunit. This modification of DG may also be unique to, or highly elevated in, cancers cells (particularly metastatic cells).

The invention thus provides anti-PTMA ligands (e.g., antibodies) that bind to epitopes on such post-translationally modified polypeptides (e.g., DG or on DG-associated molecules), that are displayed at the cell surface of cancer cells as the result of modification. Such probes can be used to detect cancerous conditions in tissue biopsies, and can be used to target chemotherapies to cancer cells in vivo by a variety of methods, including “immunoliposomes”.

For example, in a diseased cell (e.g., a malignant or pre-malignant cell), the DG protein complex is modified by extracellular factors, such as metalloproteases, into a disease-specific form. This modified form of the complex includes shedding of the α-DG portion from the cell surface, and direct cleavage of the α-DG subunit. Other modifications include hypoglycosylation of the α-dystroglycan subunit in advanced cancer cell models.

Altered glycosylation is evident on the cell-surface of other diseases besides cancers. Examples include polycythemia vera, where aberrant glycosylation of the thrombopoietin receptor is evident. Post-translational protein alterations are also induced in cells infected by pathogens. One example is the changes in posttranslational modifications of the protein “nucleolin” in lymphocytes infected by HIV. Therefore, a phenotypically derived cell can be created by pathogen infection of control cells.

The invention also provides an antigen comprising a hypo- or hyperglycosylated peptide domain compared to a normal peptide having the same sequence. For example, as demonstrated herein dystroglycan is hypoglycosylated in cancer phenotypes compared to normal non-cancer cells. A hypoglycosylated domain (epitope) of DG can be used to induce an immune response. In one aspect, the immune response provides a protective immunity to the subject. In another aspect, the hypoglycosylated antigenic peptide can be formulated as a vaccine either alone or in combination with an adjuvant.

Adjuvants are substances that can assist an immunogen in producing an immune response. Adjuvants can function by different mechanisms such as one or more of the following: increasing the antigen's biologic or immunologic half-life; improving antigen delivery to antigen-presenting cells; improving antigen processing and presentation by antigen-presenting cells; and inducing production of immunomodulatory cytokines. (Vogel, Clinical Infectious Diseases 30 (suppl. 3):S266-270, 2000.)

A variety of different types of adjuvants can be employed to assist in the production of an immune response. Examples of particular adjuvants include aluminum hydroxide, aluminum phosphate, or other salts of aluminum, calcium phosphate, DNA CpG motifs, monophosphoryl lipid A, cholera toxin, E. coli heat-labile toxin, pertussis toxin, muramyl dipeptide, Freund's incomplete adjuvant, MF59, SAF, immunostimulatory complexes, liposomes, biodegradable microspheres, saponins, nonionic block copolymers, muramyl peptide analogues, polyphosphazene, synthetic polynucleotides, IFN-gamma, IL-2, IL-12, and ISCOMS. (Vogel Clinical Infectious Diseases 30 (suppl 3):S266-270, 2000, Klein et al., Journal of Pharmaceutical Sciences 89:311-321, 2000, Rimmelzwaan et al., Vaccine 19:1180-1187, 2001, Kersten Vaccine 21:915-920, 2003, O'Hagen Curr. Drug Target Infect. Disord., 1:273-286, 2001).

In one aspect, the invention provides an antigen comprising an α-DG extracellular domain epitope that is hypoglycosylated compared to a normal domain. The domain comprises the mucin-like region, which is readily identifiable in the art. In one aspect, the α-DG domain useful as an antigen comprises a laminin 2 binding domain. Such hypoglycosylation may be chemically, enzymatically or genetically engineered. For example, GlycoPro exo-glucosidase glycoprotein removal kit (ProZyme, San Leandro, Calif., USA) may be used to treat a DG domain to remove glycosylation. N-glycosylation sites in eukaryotic polypeptides are characterized by an amino acid triplet Asn-X-Y, wherein X is any amino acid except Pro and Y is Ser or Thr. Appropriate modifications to the nucleotide sequence encoding this triplet will result in substitutions, additions or deletions that prevent attachment of carbohydrate residues at the Asn side chain. Alteration of a single nucleotide, chosen so that Asn is replaced by a different amino acid, for example, is sufficient to inactivate an N-glycosylation site. Known procedures for inactivating N-glycosylation sites in proteins include those described in U.S. Pat. No. 5,071,972 and EP 276,846. Mutation of Asn (N) 141 to Glutamate (E) in human DG .creates a hypoglycosylated DG on the cell surface. Accordingly, antigens useful in the invention can be recombinantly produced.

The invention further provides certain cell lines that express precursor molecules that can be induced to change into phenotypically derived markers. For example, cell lines expressing normal types of dystroglycan can be induced to modify these normal types into malignant types by incubating cells with a modifying agent (e.g., TGF-β and/or a metalloprotease). Conversely, a cell line displaying a disease specific marker can be treated to restore the protein structure existing in normal tissues (e.g., using a protease inhibitor). Thus, two cell lines are enabled which are essentially similar except that one displays the normal marker (precursor antigen), and the other the phenotypically derived diseased antigen (PTMA-antigen).

The invention provides for methods for screening for moieties comprising post-translational modification, i.e., PTMA, and for anti-PTMA ligands. The invention further teaches a method of identifying a ligand that binds to a PTMA comprising the steps of: (a) contacting a ligand library having a plurality of members with cells that display the PTMA; (b) separating the cells from the library members that do not associate with the cells. The library members that are associated with the cells may be further isolated, and optionally, propagated or amplified, and subjected to subsequent repetitions of steps (a)-(b). In another embodiment, the invention teaches a method for identifying ligands directed to a marker, e.g., a PTMA, phenotypically derived from a precursor, comprising the steps of:

(1) contacting a ligand library having a plurality of members with cells of a first cell line that displays a precursor antigen;

(2) separating the ligand library members that do not associate with the cells of the first cell line; and

(3) contacting the ligand library members separated in (2) with the cell of the second cell line, wherein the second cell line is obtained by a process comprising inducing the cells of the first cell line to modify the precursor antigen into the phenotypically derived marker (e.g., the PTMA).

The second method holds an advantage of having the library depleted from any members that bind to epitopes which are present on the cell surface, other than the sought marker (e.g., PTMA), since these other epitopes are likely to be common for both first and second cell type. Then the members of the library that bind to the modified cells would predominantly be those that bind specifically to the sought marker. In one preferred embodiment a ligand library is a ligand display library, such as a bacteriophage (phage) display library.

A ligand library is a group, or pool, of randomly or partially randomly derived chemical or biological entities, at least some of which are expected to be capable of binding a target antigen. These chemical or biological entities may be present either alone or displayed on the surface of a carrier; including, but not limited to, yeast, viruses, and beads. In the latter case a ligand library is referred to as a ligand display library. Libraries may include, but are not limited to phage, yeast, viral in general, nucleic acid, beads, chemical, and peptide Ligands may include peptides, proteins, such as antibodies, antibody fragments, single chain antibodies, single domain antibodies, peptidomimetics, aptamers, oligonucleotides, small molecules, or any molecule capable of binding to a target antigen.

Alternatively, affinity matured ligands may be selected using this approach. Ligands can be modified at selective sites (antigen binding domains on antibodies for example) deemed important for binding and then selected for improved binding or internalization. The invention also describes the situation where these matured ligands are screened and selected.

There is a plurality of approaches for preparing the combination of phenotypically-derived and control cell lines The control cell lines, utilized in the contacting step (1), above, also referred to as the first cell line, can be immortalized cell lines, primary cultures, or tissue explants, or intact tissues. They can originate from benign or malignant tissues, embryonic or adult. They can originate from a mammalian species, or from non-mammalian cells or from any cell that expresses a given protein by transgenesis. The control need not be intact cells, but can also be proteins isolated from control cells, or proteins expressed through an in vitro translation method. In the example of dystroglycan modifications (described herein), the control cell line is one that expresses forms of dystroglycan and/or dystroglycan-associated proteins, present on normal cells in vivo.

Contacting of the library members with the cells is performed for a time, and under the conditions, sufficient for the library members that have affinity to the cells to become physically associated (e.g., bound or internalized) with the cells, as described, for example in U.S. Pat. No. 6,794,128 and Barry et al., 1996, Nature Medicine, vol. 2, p, 299-305. Such contacting may be, for example, by co-incubation. Co-incubation of the library members with the cells of the first cell line may be at low (e.g., 4° C.) or normal body temperature, 37° C. In the first case only non-specifically binding library members will be removed, in the second case, non-specifically binding and internalizing members will be removed. The cells may be adherent or in suspension. If the cells are adherent, the separating step may be simply decanting the supernatant liquid containing non-cell bound library members. If the first cells are in suspension, centrifugation or filtration can be used. The separated library members can be used directly or amplified.

The second cell line is generated, for example, by cultivating the cells of the first line in the presence of a modifying agent that induced modification of the precursor antigen into a phenotypically derived antigen/marker. Such modifying agent may include changes in temperature, acidity, redox potential, hypoxia, subjecting the cells to light or ionizing radiation, or to a chemical factor, or infection by a pathogen (e.g., HIV). It may also include modification by enzymes, including, but not limited to, proteases, glycosidases, kinases, phosphatases, sulfatases, and lipid transferases, such as farnesyltransferase, myristoyl transferease, and palmitoyl transferase. It may also include the expression of regulatory proteins, such as oncogenes, that alter the expression of aforementioned enzymes or modify enzymes to alter their activity. It may also include enzymes that directly modify the molecule to be targeted, resulting in altered protein folding, the destabilization of a protein complex, new exposure of protein residues, or rendering the protein accessible to modifying enzymes like proteases. Thus, in the case of dystroglycan, glycosidases and peptidases appear to play a role in posttranslational modification, and the exposure of novel epitopes. In this situation, a control cell line could be a cell line grown in the presence of glycosidase or protease inhibitors and the phenotypically derived cell line grown in the absence of such inhibitors. For example, a metalloprotease cleaves β-dystroglycan at the cell surface, and a metalloprotease inhibitor (e.g., GM6001 or BB2516) can be used block this cleavage event. Alternatively, one could also create the pairs of modified and unmodified proteins by adding or removing an activator of the enzyme responsible for the post-translational modification. For example, treatment of cells with phorbol esters induces metalloprotease activity. Some proteases can cleave the proprotein domain of other proteases, leading to enzyme activation through a protease cascade.

Alternatively, as also described for dystroglycan, one can affect the modification of the target antigen indirectly by affecting interactions with other proteins either upstream or downstream in a signal transduction pathway. For example, the addition of TGF-β results in destabilization of the dystroglycan protein complex and the appearance of new epitopes in dystroglycan and dystroglycan-associated proteins. Expression of the Ras oncogene also results in the destabilization of the dystroglycan protein complex, and exposure of new epitopes in dystroglycan and dystroglycan-associated proteins.

It is useful to establish whether the modification took place and the second cell line was indeed obtained. For example, it can be done by analyzing the presence of the precursor and the modified factor by known methods such as Western blotting, electrophoresis, immunoprecipitation, chromatography, ELISA, lectin affinity, and mass spectrometry. For example, in cells expressing dystroglycan, the modification of these proteins is evidenced by the loss of detection of a particular component, and by shifts in their molecular masses. For example, shedding of α-dystroglycan is evidenced by the decrease in the ratio of α to β-dystroglycan detected in cell extracts; a decrease in this ratio indicates a selective loss of α-dystroglycan from the cell surface. The increased detection of α-dystroglycan in the cell culture medium also measures α-dystroglycan shedding from the cell surface. Cleavage of the β-dystroglycan subunit is evidenced by a shift in the molecular mass of this protein from 43 kDa to 31 kDa, as detected by immunoblotting with an antibody binding the β-dystroglycan cytoplasmic domain. Altered glycosylation of α-dystroglycan is evidenced by a shift in the molecule mass of this subunit from the normal mass (150 kDa) to the abnormal mass (100 kDa). This shift in mass is again detected by immunoblotting using anti-α-dystroglycan antibodies, or using streptavidin binding of immunoprecipitated proteins of the dystroglycan complex following cell-surface biotinylation. Also, altered glycosylation of α-dystroglycan is evidenced by the binding (or absence thereof) of anti-dystroglycan antibodies that depend on certain carbohydrate moieties for binding (e.g., the IIH6 and VIA4 antibodies).

The members of the library, for example, separated from the cells of the first cell line in step (2), above, can then be contacted with cells of the line that displays the PTMA of interest, such as the cells of the second cell line. The contacting may be conducted by co-incubation at internalization-permitting conditions, for example, 37° C. and the source of energy for the cells, or under internalization-inhibiting conditions, such as the temperature below 37° C., typically at 4° C., or in the presence of metabolic inhibitors like sodium azide or deoxyglucose. The library members that associate with the cells are then separated by separating the cells and optionally, washing them to remove unbound library members with physiological salt buffers at pH near neutral (typically pH 6-8). The library members can be then eluted from the surface of the cells, for example, by action of a competing ligand, or by action of high ionic strength (0.5-2M NaCl), low pH (pH 2-4), or in the presence of 0.1-6M urea. If the task is to obtain internalizable library members, the cells may be first washed under any of these surface-elution conditions, and the internalized library members can be recovered from the cells after mechanical disruption or lysis of the cells by any known methods.

During contacting the library members are typically recovered, and the ligands they bear are identified. Methods for identification of ligands following selection of ligand display libraries are known in the art. Optionally, the recovered ligand display library members can be subjected to (1)-(4) (as described above) one or more times to further increase the specificity of selected ligands.

The ligands so produced have higher degree of specificity to phenotypically produced markers than the ligands produced by simple positive selection of steps (a)-(b), above, or by immunization/hybridoma methods that do not eliminate non-specifically binding ligand clones.

In addition to using cell lines to identify or select for binding ligands to disease-specific or overexpressing antigens, one can also utilized purified proteins, peptides, or sugars when the modification is reasonably well understood. For example, in the case of dystroglycan, there is some evidence that αDG subunit may simply be lost from the cell surface due to disruption of interactions between the alpha and beta subunits. Thus a protein corresponding to the extracellular domain of βDG that becomes exposed may be used to coat a plate and select against. Binding has been the single greatest selection criteria used to date and thus provides a reasonable alternative to cell-based screening in situations where functional screening is not essential. Other examples include the use of peptides or sugars corresponding to the post-translationally modified regions of the target antigen/epitope when the modification is well understood. Purified proteins can also be treated with enzymes such as glycosidases and proteases to induce the appearance of the new epitope in vitro, which can be subsequently selected against.

These ligands can be used for example as diagnostics or targeted drug/gene delivery systems. These ligands may have useful biological effects, such as malignancy growth prediction and/or reversal of malignant phenotype. Ligands can be used to deliver a variety of biologically active agents or diagnostic agents. These include, but are not limited to, free drug or imaging agents, radioisotopes, toxins, or agents encapsulated, complexed or bound to a variety of drug delivery systems (liposomes, nanoparticles, viruses, polymers, and the like). For example, drug-loaded liposomes targeted with either scFv (Nielsen et al., 2001) or Fab′ (Kirpotin et al., 1997; Park et al., 2001) antibody fragments have shown considerably increased activity in cell culture using cytotoxicity assays and also in vivo in antitumor efficacy studies.

Selecting against post-translationally-modified antigens allows for the identification of targeting ligands to novel disease-specific epitopes. The use of disease-specific epitopes are a useful embodiment of this invention. However, it is often valuable to have ligands that are targeted to antigens that are merely overexpressed on a diseased tissue. For example, HER2/neu is overexpressed on certain aggressive cancers, but is not specifically expressed on these cancers. It is also found in cardiac muscle tissue, allowing for a potential site of toxicity for therapies targeted to this antigen. However, both Herceptin and HER2-targeted immunoliposomes display considerable activity and relatively moderate toxicity despite this distribution of expression. In addition, HER2-targeted immunoliposomes have demonstrated a threshold effect for receptor-mediated internalization, and thus activity, whereby at low antigen densities such as found in MCF7 breast cancer cells (<10,000 receptors/cell) internalization is essentially the same as nontargeted liposomes, and cells expressing moderate-to-high levels of the antigen (>100,000 receptors/cell) demonstrate rapid internalization and specific activity. This suggests that higher expression and not necessarily specific expression can be adequate for significant disease inhibiting activity.

The use of non-immunogenic ligand libraries, such as human antibody phage display libraries, allows for the targeted diagnostics or therapeutics to be administered multiple times without concern for increased clearance from the general circulation upon repeat administration. Thus, one embodiment of this invention includes the use of such non-immunogenic libraries.

Ligands are historically most often selected based on binding affinity. Weak or nonspecific binders are removed and tight binders are collected, amplified, and reselected. However, the invention demonstrates that for some applications, internalization is as important a criteria for selection as binding, if not more important. Weak binding ligands may actually be used due to their reduced capacity to be limited by the binding site barrier present upon entering some diseased tissues such as solid tumors. In addition, therapeutics directed using weak binders may be less affected by shed antigen present in the blood. Finally, increased binding does not appear to necessarily correlate with increased internalization. Indeed, the scFv termed F5 binds considerably weaker than C6.5, but internalizes more efficiently resulting in a better therapeutic activity. HER2-targeted liposomal doxorubicin is relatively ineffective when targeted using a tight binding. Methods for selecting antibodies based on internalization are known (see, e.g., U.S. Pat. No. 6,794,128). In this method non-internalized antibodies are stripped from the membrane surface using and acid or EDTA wash and the internalized phage subsequently recovered following cell lysis. Accordingly, one embodiment of the invention involves selecting antibodies for internalization via post-translationally modified receptors or cell surface proteins.

The incorporation of internalization criteria in the screens results in the identification and production of antibodies with a high potential utility for liposome-targeting of therapeutics to sites of disease. In the one aspect of the invention, non-internalized phages are stripped from the membrane surface, for example, using and acid wash, optionally with proteolytic reduction of extracellular matrix, for example, by trypsinization, and the internalized phage subsequently recovered following the cell lysis. Nonspecific phages are removed via negative selections on control cell lines. The invention involves adapting this method by selecting antibodies for internalization via post-translationally modified receptors.

The invention provides methods and compositions of an expanded arsenal of antibodies that bind to tumor-specific antigens and mediate internalization. Although the antibodies currently in use are proving effective, the few targets investigated to date are neither optimal nor sufficient. An expanded arsenal of antibodies is necessary to target the full diversity of cancers that arise in human subjects, and to permit the customized treatment of individual cancers; antibodies obtained that efficiently target cancers of different origin, and antibodies obtained that target the variations evident among distinct classes and grades of cancer.

As mentioned above, one approach to the targeting of cell surface antigens on cancer cells is the immunotargeting of liposomes to cancer cells. Liposome vesicles can be loaded with various chemotherapeutic drugs using gradient-based drug loading strategies. Chemotherapeutics that can be loaded into liposomes include, for example, anthracyclines, vinca alkaloids such as vinorelbine and vincristine, and the camptothecins, irinotecan and topotecan. These liposomes are stable in circulation and permit only limited release of the drug from the liposome while in the general circulation. Therefore, high doses of drugs that might even be toxic to normal tissue when administered in the free form, can be administered comparatively more safely when encapsulated in liposome vesicles. The targeting of liposomes to cancer cells requires escape from the vasculature, binding of the liposome to the cancer cell surface, and subsequent internalization (endocytosis) of the vesicle. Once inside the cell, the liposome is degraded and the drug released to work directly on the cancer cell, and often neighboring cells via a bystander effect. Methods and compositions related to liposomal drug delivery are well known in the art and can be used to practice the invention. See, for example, Liposomes. Methods in Enzymology, vols. 376 (2003), 372 (2003), 373 (2003), 387 (2004), and 391 (2005).

Accordingly, the invention provides for screening of a phage display antibody library to generate human single-chain antibodies directed to post-translational protein modifications at the surface of cancer cells. In one embodiment, two forms of protein modifications are targeted: 1) metalloprotease-induced protein cleavage events and 2) altered glycosylation of specific glycoproteins. Both of these modifications are evident at the surface of cancer cells. The activation and expression of metalloproteases are enhanced in the tumor microenvironment, originating from the cancer cells themselves and from the surrounding stroma. Cell surface proteins known to be modified by metalloproteases include, for example, E-cadherin, syndecan-1 and -4, CD44, MT1-MMP, L1, nectin, DDR1, dystroglycan, EGFR ligands (proTGF-β, promphiregulin and proepiregulin, pro-HB-EGF), Her-2/neu, MUC1. Other cell-surface proteins modified by metalloproteases, and other cleavage events, known and those yet to be identified, are also within the scope of this invention. There are also many known alterations in the carbohydrate composition of cell-surface glycoproteins, which are useful in practicing the invention. These include, without limitation, CD44, 1-integrin, MUC 1 and Ep-CAM, syndecans, and dystroglycan.

A high diversity phage display library is useful for successfully selecting antibodies against most antigens/targets. In general, a large phage antibody library requires at least 10⁹ independent clones for screening. Such libraries are made by carrying out a large number of ligations and transfections. The invention provides a high diversity phage-display human scFv antibody library that include two major innovations. A phagemid antibody display vector with incorporated lox Cre recombination sites in the linker portion between the light and heavy chains for intracellular recombination within bacterial host to transform a primary library of diversity of 7×10⁷ members into a secondary library of diversity of 3×10¹¹. See, for example, PCT Pat. Appl. PCT/EP99/08856.

In one embodiment, the high diversity phagemid library is prepared from the primary library by infecting M13KO7 helper phage with the primary library bacteria and selecting with kanamycin. The phagemid library is then isolated by centrifugation in the presence of PEG/NaCl. A large secondary library is produced using intracellular recombination to improve the diversity of the primary library. The primary phagemid library is added to E. coli bacteria constitutively expressing Cre recombinase to allow infection to occur. Helper phage are later added and the secondary phagemid library isolated using the PEG/NaCl centrifugation method. Phenotype and genotype are then coupled by reinfection DH5αF′ E. coli with phagemid harvested from the supernatant of the PEG/NaCl method.

The invention also provides a cancer antigen comprising a modified DG. In addition, the invention provides an anti-PTMA-DG composition capable of targeting a biologically active agent or diagnostic to a cell expressing a post-translationally modified DG.

The invention demonstrates that hypoglycosylated forms of dystroglycan (which lacks binding properties to known ligands) contribute to cancer progression through novel and previously unrecognized functions. The modified DG arises as the result of normal signaling processes that are corrupted or exaggerated in cancer cells; 2) contains functions that are distinct from the laminin binding isoform; and 3) itself imparts cellular changes that are evident in cancer cells and are believed to aid in cancer progression. Thus, an agent that binds specifically to this hypoglycosylated isoform of dystroglycan, can perturb the functions of the hypoglycosylated dystroglycan that promote cancer progression, and thereby prove therapeutic in the treatment of cancers.

The hypoglycosylated isoform of DG is generated by cooperative signaling through the TGF-beta and Ras/MAPK pathways in functionally normal cells. Thus, the creation of this isoforms is the result of a normal regulatory mechanism in cells, suggesting that the molecule serves some function itself in normal cell biology. Because the TGF-beta and Ras/MAPK pathways are misregulated in cancers, the hypoglycosylated isoforms of DG arises from corruption of these normal signaling networks.

The current dogma about DG function assumes that the hypoglycosylated form of DG (lacking known ligand binding properties) is inactive on the cell surface. An alternative hypothesis is that the hypoglycosylated DG imparts functions that are distinct from the more commonly expressed laminin-binding isoform. Through site-directed mutagenesis of dystroglycan, a hypoglycosylated DG molecule was expressed in mammary epithelial cells lacking endogenous DG expression. The gene expression profile of these cells was obtained by Affymetrix microarray analysis and compared to that of cells lacking any DG expression (DG−/− cells) and those expressing the normal, laminin-binding isoforms of DG (wtDG). The expression profiles are each unique, demonstrating that the hypoglycosylated DG isoform does indeed impart signals in the cell, and many of these signals are distinct from those the laminin-binding DG isoforms.

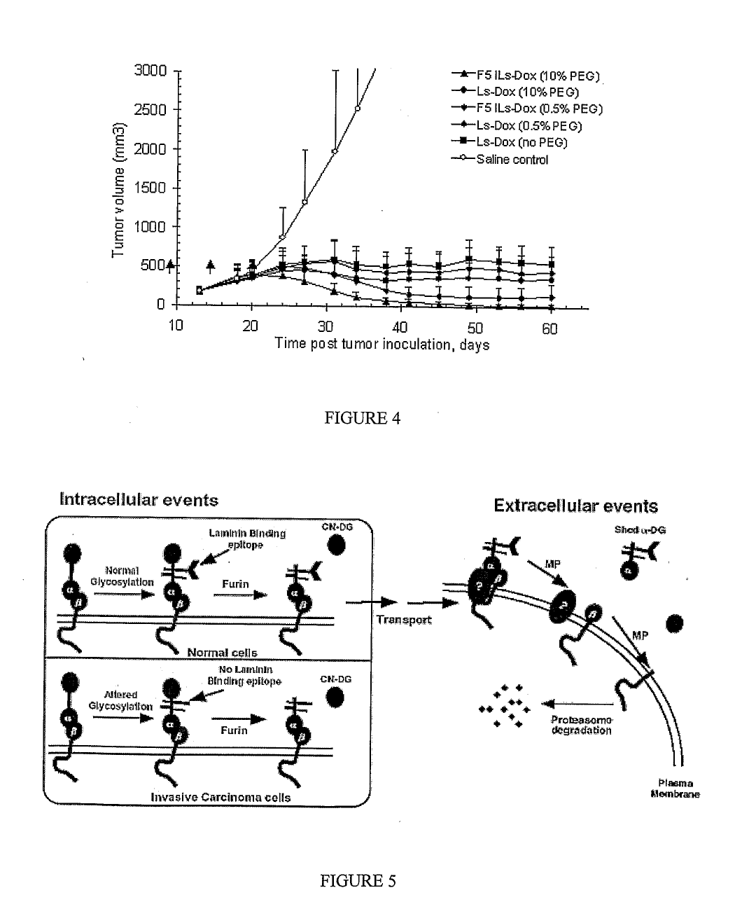

From the microarray analysis, several genes were identified that are specifically regulated by expression of the hypoglycosylated DG isoform. Notable among these genes is a strong upregulation of the cell-surface molecule N-cadherin, a homophillic cell adhesion molecule. N-cadherin is not normally expressed in epithelial cells, but elevated N-cadherin expression is observed in carcinoma progression, and the aberrant expression of this cell adhesion molecule is believed to enhance tumor cell invasion. Therefore, expression of the hypoglycosylated form of DG in epithelial cells imparts cellular changes that facilitate cancer progression.

Where studied (EGFR, HER2), molecular targeting resulted in increased in vitro cytotoxicity and in vivo antitumor efficacy. (Park, et al., 2002) An example of the observed efficacy is shown in FIG. 4. Although, poly(ethylene glycol)-coated (PEGylated) liposomal doxorubicin formulations were effective in controlling of the tumor growth, HER2(F5)-immunoliposomal doxorubicin was shown to result in significant tumor regressions and even complete remissions in more than 50% of the animals. Similar results with a wide range of different liposomal therapeutic agents, including vinorelbine, vincristine, topotecan, epirubicin, and doxorubicin have been observed. It is understood that while liposomes are useful, a wide range of pharmaceutical agents can be used to practice the invention, such as cytotoxins (the agents that in pharmacologically acceptable doses reduce the rate of cell proliferation and/or cause cell death), various pharmaceutically active molecules, drug carriers (such as polymers or dendrimers), nanoparticles, polynucleotides (DNA, RNA, synthetic oligo- and polynucleotides), detectable markers—such as X-ray, MRI contrast agents or radioisotopes.—In addition, there are several criteria for successful drug delivery using immunotargeted liposomes, including maximizing of the liposome accumulation in the tumor due to the enhanced permeability and retention effect due to good in vivo retention of the drug by the carrier, and good longevity of the carrier in the circulation. Each of these factors has been carefully controlled to maintain liposomal particles with high stability and favorable pharmacokinetic properties, helping allow for maximum efficacy when targeted with specific antibody fragments.

A chimeric molecule (e.g., a targeting ligand that specifically binds to a post-translationally modified polypeptide linked to a biological active agent or label) can be formulated for use parenterally, topically, orally, or locally for prophylactic and/or therapeutic treatment. The pharmaceutical compositions can be administered in a variety of unit dosage forms depending upon the method of administration. For example, unit dosage forms suitable for oral administration include powder, tablets, pills, capsules and lozenges. It is recognized that a chimeric molecule or pharmaceutical compositions of this invention, when administered orally, must be protected from digestion. This is typically accomplished either by complexing the molecule with a composition to render it resistant to acidic and enzymatic hydrolysis or by packaging the protein in an appropriately resistant carrier such as a liposome. Means of protecting molecules from digestion are known in the art.

The pharmaceutical compositions of the invention are particularly useful for parenteral administration, such as intravenous administration or administration into a body cavity or lumen of an organ. The compositions for administration will commonly comprise a solution of the chimeric molecule dissolved in a pharmaceutically acceptable carrier, typically an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of chimeric molecule in these formulations can vary widely, and will be selected primarily based on fluid volumes, viscosities, body weight and the like in accordance with the particular mode of administration selected and the patient's needs.

Thus, a typical pharmaceutical composition for intravenous administration would be about 0.1 to 100 mg per patient per day. Dosages from 0.1 up to about 1000 mg per patient per day may be used, particularly when the drug is administered via slow infusion, or to a secluded site and not into the blood stream, such as into a body cavity or into a lumen of an organ. Methods for preparing parenterally administrable compositions will be known or apparent to those skilled in the art and are described in more detail in such publications as Remington's Pharmaceutical Science, 15th ed., Mack Publishing Company, Easton, Pa. (1980).

The compositions containing a binding ligand linked to a biologically active agent or diagnostic (e.g. a chimeric agent) can be administered for therapeutic treatments. In therapeutic applications, compositions are administered to a patient suffering from a disease, typically a cell proliferative disorder, in an amount sufficient to cure or at least partially arrest the disease and its complications. An amount adequate to accomplish this is defined as a “therapeutically effective dose.” Amounts effective for this use will depend upon the severity of the disease and the general state of the patient's health.

Single or multiple administrations of the compositions may be administered depending on the dosage and frequency as required and tolerated by the patient. In any event, the composition should provide a sufficient quantity of the proteins of this invention to effectively treat the patient.

Among various uses of the cytotoxic chimeric agents of the invention are included a variety of disease conditions caused by specific human cells that may be eliminated by the toxic action of a chimeric agent. One application is the treatment of cancer, such as by the use of an antibody attached to a cytotoxin.

Another approach involves using a ligand that binds a cell surface marker (receptor) so the chimeric agent associates with cells bearing the ligand substrate are associated with the post-translationally modified cell surface polypeptide on a tumor cell.

In another embodiment, this invention provides kits for the treatment of cell proliferative diseases or disorders or for the detection of cells comprising a post-translational isoform of a cell surface polypeptide, e.g., a PTMA. Kits will typically comprise a chimeric molecule of the invention (e.g. antibody-label, antibody-cytotoxin, antibody-ligand, etc.). In addition the kits will typically include instructional materials disclosing means of use of chimeric molecule (e.g. as a cytotoxin, for detection of tumor cells, to augment an immune response, etc.). The kits may also include additional components to facilitate the particular application for which the kit is designed. Through the use of diagnostic articles comprising the ligands of the invention, the patients with appropriate levels of PTMA abundance and therefore, higher likelihood of benefit from the treatment with the invented therapeutic compositions, are identified. The following examples are intended to illustrate but not limit the disclosure. While they are typical of those that might be used, other procedures known to those skilled in the art may alternatively be used.

### EXAMPLES

Generating cell-internalizable human single-chain Fv antibodies specific to post-translationally modified proteins in breast carcinoma cells. Starting from a phage display human scFv antibody library, the phage clones that express scFv antibodies binding post-translationally modified proteins specifically presented on breast carcinoma cells are identified and isolated. In particular, the PTMA include metalloprotease-induced modifications of proteins at the surface of breast carcinoma cells, and epitopes that arise from altered glycosylation of a-dystroglycan (a-DG). The screens employ live cells and the methods that permit concurrent selection for antibodies that mediate phage internalization subsequent to cell-surface binding, is a potential advantage for use of these selected scFvs as targeting ligands for intracellular delivery of the nanocarriers.

To achieve selectivity for PTMA in live cell screens, the screening cycle includes two steps. In the first (negative selection) step a “precursor”, or subtractive, cell population carrying a non-modified antigen is used to deplete the library of the non-specifically reactive phages or those that react with epitopes other than the epitope of interest. The second (positive selection) step uses the precursor cell line modified to induce the post-translational modification of interest, and the resulting “modified” cell population, which will be used to select for those phage that bind to the protein modification of interest. Additionally, the phage internalization/surface stripping is employed at the second step to isolate the phages that carry internalizing anti-PTMA antibodies. The method used to create the precursor and modified cell population thus determines the modifications to be targeted.

Screen for antibodies binding to metalloprotease-induced modifications of proteins at the surface of breast carcinoma cells. Protein modifications by metalloprotease action are enhanced in the environment of cancer cells, and these include modifications of many cell-surface molecules (Egeblad and Werb, 2002). To target metalloprotease-induced modifications at the cancer cell surface, a highly diverse phage display antibody library will be screened by selecting for differential adhesion and internalization in cancer cells cultured in the presence and absence of a broad-spectrum metalloprotease inhibitor, the former population being depleted of metalloprotease-induced modifications. This method is designed to capture antibodies against any metalloprotease-induced neoepitopes arising at the cell surface.

Screens for antibodies binding to metalloprotease-induced protein modifications on cancer cells will use living cells bearing such neoepitopes on the cell surface. Cultured human breast carcinoma cell lines is employed as a convenient and useful source, expressing many endogenous metalloproteases. Two different human breast carcinoma cell lines, MDA-MB-231 and BT474, are employed in separate screens for antibodies binding to metalloprotease-induced neoepitopes. Both cell lines originated from human metastatic breast cancers, but each represents a distinct cancer cell phenotype. The MDA-MB-231 cells are highly aggressive in xenograft assays of tumor growth and metastasis (Lacroix and Leclercq, 2004). They have mesenchymal characteristics, lack cell-cell adhesion, are estrogen and progesterone receptor (ER/PR) negative (Lacroix and Leclercq, 2004), and have lost the ability to respond appropriately to extracellular matrix molecules (Muschler et al., 2002). In contrast, the BT474 cells retain epithelial characteristics, are less aggressive in xenograft assays of cancer growth (Park et al., 2002), are (ER/PR) positive (Lacroix and Leclercq, 2004), and retain the ability to respond to certain cues from extracellular matrix molecules (Muschler et al., 2002). BT474 cells have been previously studied for in vivo tumor growth and treatment with liposomes (Park et al., 2002). By using these two very different cancer cell lines, antibodies recognizing distinct sets of tumor specific antigens, those recognizing ER/PR positive, non-aggressive, early stages cancers, and those recognizing highly invasive, ER/PR negative tumors, are isolated.

The second requirement for a successful screen is the creation of a precursor cell population that does not display the metalloprotease-induced modifications. As metalloprotease cleavage events can be easily blocked in cultured cells by the inclusion of a metalloprotease inhibitor in the culture medium, with no loss in cell viability. Therefore, by culturing a carcinoma cell line for an extended period in the presence of a broad-spectrum metalloprotease inhibitor, the precursor population depleted of metalloprotease-induced protein modifications can be created.

The metalloprotease inhibitor chosen for this application is the hydroxamate, GM6001. GM6001 has been well characterized and know for its broad specificity (Galardy et al., 1994) and is commercially available from Chemicon International (Temecula, Calif.). All cell lines tested so far, including the BT474 and MDA-MB-231 cells, can tolerate high concentrations (50 μM) of this inhibitor for extended periods of time without loss of cell viability (Singh et al., 2004), and these concentrations are more than sufficient to block the activity of known metalloproteases (Galardy et al., 1994; Singh et al., 2004).

The carcinoma cell lines are cultured in a low-serum media to subconfluence and treated for 6 days with of 50 μM GM6001, with one change of medium after two days. The precursor population (treated with GM6001) is used in a subtractive screen to absorb and eliminate phage that bind to cell-surface antigens not arising from MP activity. The untreated population displays metalloprotease-induced antigens on the cell surface and can be used in a second step of the screening cycle—cell-binding and internalization screen of the depleted phage library.

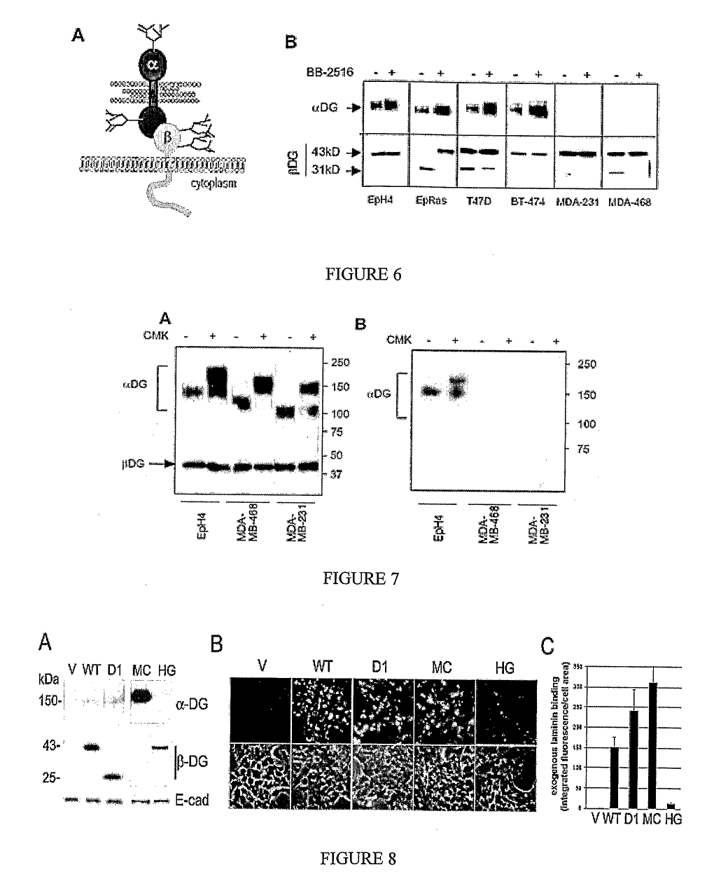

To confirm that metalloprotease activity is indeed blocked by the GM6001, the cleavage of known molecules is monitored. Using identically treated cell populations, proteins are extracted and analyzed by immunoblot. These controls include detecting the cleavage of the β-DG subunit, which is evidenced by a shift in the molecular mass of this protein from 43 kDa to 31 kDa in MDA-MB-231 cells using an antibody binding the β-DG cytoplasmic domain (see FIG. 6).

The high diversity phage display antibody library is screened according to the known methods in the art. (Paul, Marks, Becerrill, 2000). Briefly, phagemid library is propagated by infection of a permissive E. coli strain. Following lytic cycle, the phages are isolated from the bacterial lysates by PEG precipitation and quantified in a conventional manner. About 2×10¹² phage particles are first incubated with 107 adherent subtractive cells for 4 h at 4° C., with rocking. The supernatant, containing the unbound phage (the “depleted” library), will be collected from the cell culture dishes and centrifuged for 5 minutes at 1200×g to remove cells that may have detached from the dishes. This selection process will be repeated a second time for higher stringency.

The supernatant containing the depleted phage library will then be incubated with 106 adherent “modified” cells for 1 h at 4° C., the cells are washed with cold phosphate-buffered saline (PBS) and incubated with pre-warmed (37° C.) medium plus 2% fetal calf serum (FCS) at 37° C. for 30 min to allow receptor-mediated internalization. Non-internalized phage are removed by washing cells with a low-pH glycin-urea buffer, the cells are harvested by trypsinization, washed in PBS, lysed with 1 ml of 100 mM triethylamine, and neutralized with This-HCl pH 6.8. The lysate is used to infect exponentially growing E. coli TG1, as described previously (O'Connell et al., 2002), to amplify the selected library members. The recovered and amplified library members are subjected to same selection steps two more times to further increase the specificity of selected ligands.

Following three rounds of selection and amplification, the remaining phage is prepared as individual phage plaques, and isolated. The number of unique phage antibodies are determined by assaying the patterns of BstNI digestion of scFv genes amplified by PCR from phage-infected bacteria (Liu et al., 2002). When restriction digest patterns are difficult to distinguish, scFv genes are sequenced to determine their identity and uniqueness.

To select the ligands reactive to PTMA produced by metalloproteases contributed by stromal cells in the tumor microenvironment, other carcinoma cell lines are used, with the addition of conditioned medium from stromal cells, or the addition of other factors, such as the phorbol ester PMA, which can induce metalloprotease gene expression (Benbow and Brinckerhoff, 1997). However, the most likely outcome of the proposed screens is the isolation of a large number of potential agents.

Screening for antibodies binding to epitopes that arise from altered glycosylation of α-dystroglycan (DG) in breast carcinoma cells. The varied carbohydrate modifications to be targeted in this proposal are those arising on DG. DG is part of a glycoprotein complex expressed on the cell surface of all normal epithelial cells, yet hypoglycosylation of the α-DG subunit is evident in the majority of advanced breast carcinoma cell models and have been detected in other carcinomas as well (Singh et al., 2004). This modification causes complete loss of receptor function, and may contribute to the progression of the disease (Singh et al., 2004). The targeting of DG modifications is of high interest for several reasons. First, there is evidence that the identified post-translational modifications of DG are specific to or highly enriched on the tumor cell surface. Second, evidence exists that DG is likely to mediate internalization of an attached liposome: DG has been identified as the receptor for several pathogens, mediating cell infection by lymphocytic choriomeningitis virus and Lassa fever virus viruses and Mycobacterium leprae (Cao et al., 1998; Rambukkana et al., 1998), demonstrating that an exogenous agent binding to DG is likely to be internalized.

The phage display library is screened for antibodies binding to the hypoglycosylated isoform of DG, which is the predominant isoform presented on advanced breast carcinoma cells (see introduction). To achieve this screen, engineered cell lines are employed, which include a DG−/− mammary epithelial cell line and the same cell line expressing the wild type human DG cDNA and expressing a mutant isoform of the human DG protein which is hypoglycosylated, named “HG-DG”.

The wild type DG cells are used as the precursor population to deplete the phage display library of scFvs that bind to wild-type DG and to other cell-surface proteins. The HG-DG cells are then used as the modified population to bind and isolate phage that bind to the hypoglycosylated form of the human α-DG subunit.

The phage screening conditions are the same as described in detail above. Briefly, the wild type DG-expressing cell lines is used as the precursor population to deplete the phage library of nonspecific binders. This is achieved by incubating 2×10¹² phage particles with 10⁷ adherent cells for 4 h at about 4° C., with rocking. The supernatant, containing the unbound phage, is collected from the cell culture dishes and centrifuged to remove cells that may have detached from the dishes. This selection process is repeated one or more times for higher stringency.

The supernatant containing the depleted phage library is then be incubated with the “modified” (HG-DG) cells to select for phage that bind the hypoglycosylated isoforms of α-DG. The supernatant is incubated with 106 adherent cells for 1 h at 4° C. in a single 10 cm dish. Following this incubation the cells are washed with cold PBS and incubated with pre-warmed (37° C.) medium plus 2% FCS at 37° C. for 30 min to allow receptor-mediated internalization. Non-internalized phage is removed by washing cells with the phage-stripping buffer and by digesting cells with trypsin at 37° C. for 10 min. Cells are collected by centrifugation, washed in PBS, lysed with 1 ml of 100 mM triethylamine, and the lyzate neutralized. The phage-bearing lysate is then used to infect exponentially growing E. coli TG1 to amplify the selected library members. The recovered and amplified library members are subjected to same selection steps one or more times to further increase the specificity of selected ligands. Finally, the remaining phage will be prepared as individual phage plaques, and isolated. The number of unique phage antibodies is determined by patterns of BstNI digestion of scFv genes amplified by PCR from phage-infected bacteria (Liu et al., 2002). When restriction digestion patterns are ambiguous, scFv genes are sequenced to determine their identity and uniqueness.

As an alternative approach to target DG modifications on human carcinoma cells, screens for antibodies against DG isoforms are be employed using isolated proteins transferred to PVDF membranes [complete method described in (Liu et al., 2002)]. In this screen, the DG molecules of normal epithelial cells and carcinoma cells are isolated by immunoprecipitation using an antibody directed against the P-DG subunit, as described in Singh et al., 2004. The isolated proteins are separated by SDS-PAGE and transferred to PVDF membranes by standard immunoblotting methods. The portion of the membranes bearing the α-DG subunit is cut away and blocked by incubation with bovine 5% milk proteins in phosphate buffered saline (PBS). The filter bearing α-DG isolated from normal (unmodified) cells is incubated with the phage library to deplete the phage display library of scFvs that bind to epitopes common to normal cells. The non-binding members of the phage library are decanted and incubated with the membrane bearing α-DG molecules isolated from carcinoma cells. Finally, the phage bearing the scFvs that bind to modified epitopes on DG is eluted from the membrane using 100 mM triethylamine, neutralized and propagated, and the process repeated. Cell internalization screens, as described below, (Nielsen, Marks, Kirpotin, 2000) using these same carcinoma cells can be applied subsequent to this selection.

Characterizing the generated anti-PTMA scFvs for their utility as targeting ligands for tumor-targeted nanocarriers. The utility of the scFvs will be determined by several criteria including their stability, their ability to mediate internalization, and their ability to distinguish normal cells from tumor cells. The antibody-displaying phage will be converted to recombinant scFvs and screened for each of these criteria, and those targets deemed useful will be characterized.

Generating recombinant scFvs and determine their ability to mediate internalization, thermostability, and binding to IgG-specific affinity resins. Following several rounds of selection, recombinant scFvs are generated from the isolated phage and characterized by flow cytometry for cell binding and internalization, for thermostability, and finally for binding to affinity resins that bind IgG, such as Protein A and Protein G Sepharose. Cell internalization and thermostability are beneficial for liposome-mediated drug delivery, while protein A or similar affinity resin binding is beneficial for subsequent large-scale purification of the recombinant scFv.

Purified recombinant scFv may be produced, for example, produced according to Liu et al (Liu et al., 2004). Briefly, the scFv gene is subcloned from the phage vector into the secretion vector pUC119mycHis, resulting in the addition of a c-myc epitope tag and hexahistidine tag at the C-terminus of the scFv. Recombinant proteins are extracted and purified, for example, by metal chelation chromatography using the Ni-NTA carrier resin (Qiagen, Valencia, Calif.) and the purity is checked by SDS-PAGE.

In order to choose among the selected library members for the antibody clones that have the highest capacity to cause liposome binding and internalization into cancer cells, the CLIA assay of Nielsen et al. (U.S. Pat. No. 7,045,283, incorporated herein by reference) can be used as a method for identifying cell-binding and internalizing ligands, identifying receptors that are capable of internalizing ligands, and screening for antibody internalization. The CLIA assay uses a special test article, fluorescent-labeled liposomes containing nickel-chelating nitrilotriacetic acid groups attached to its surface (Ni-NTA-liposomes). Recombinant proteins such as antibodies are expressed having a hexahistidine sequence (His-tag) used to facilitate their purification. In the presence of Ni-NTA liposomes, His-tagged proteins bind to them via a non-covalent heterodentate chelation bond, instantly producing an antibody-bound liposome. When such liposomes are incubated with live cells in culture, the liposome that carry internalizable His-tagged antibodies, internalize; those that carry the antibodies that only bind, but do not internalize, remain on the cell surface. After removal of non-bound liposomes, the amount of cell-associated fluorescence will originate from both internalized and surface-bound liposomes, allowing us to screen for antibodies that interact with the cells. However, treatment of the cells with a nickel-chelating agent, such as mM imidazole, or EDTA, will dissociate the surface-bound liposomes from the antibodies, leaving behind only the internalized one. Thus, upon EDTA or imidazole treatment, the cell fluorescence will reflect the extent of internalization. By comparing the fluorescence uptake signals produced in the presence of Ni-NTA liposomes and media containing various His-tagged antibody clones, under the different washing conditions (e.g., with or without EDTA), the clones with maximum binding and/or internalization into these cells can be selected. The CLIA method is designed to be used in a 96-well plate format for high throughput screening.

In the case of breast cancer, cell binding and internalization is conveniently assayed in one of the two human breast carcinoma cell lines, MDA-MB-231 and BT474 cells, because, having been used for the initial screening, these are certain to express the antigens. The MDA-MB-231 cells also express the hypoglycosylated form of α-DG (FIG. 7). The cells (10⁶) are incubated with the scFv-bearing Chol-NTA-Ni liposomes and analyzed using a fluorescence plate reader (Biotek). Thermostability of the ScFv is assessed by first incubating the ScFv at 60 degrees Celsius for 30 minutes prior to testing cell binding and internalization as described above. The thermally stable species that retain the binding and/or internalization capacity are identified.

The binding to affinity purification ligands can be assessed by a dot blot method exemplified herein for the case of Protein A. The scFvs are pipetted onto a PVDF membrane (Immobilon-P, Millipore) the membrane is blocked for 2 hours using 5% dried milk, and then probed using Protein A-HRP, purchased from Upstate (Charlottesville, Va.). Relative Protein A binding is revealed by quantitative chemiluminescence imaging, and compared to existing positive and negative controls.

Determining the molecular identity of the targeted antigen. Subsequent utilization of the selected phage display antibodies (e.g. immunohistochemistry, affinity isolation of antigens and scFv incorporation into liposome constructs) benefits from generating purified recombinant scFvs, which is achieved by any routine methods known in the art. While the antigen is likely known for antibodies obtained in the screen against the hypoglycosylated α-DG, those obtained in the screen for metalloprotease-induced neoepitopes may be unknown. Identification of the antigen will thus be an important step in characterizing the events that give rise to the antigen and the potential consequence of its cleavage, and could help in assessing the potential usefulness of the antigen for liposome-mediated drug delivery. Therefore, potentially useful scFvs isolated above, are used to identify and characterize the antigen itself by immunoblot, immunoaffinity purification, immunoprecipitation, and/or mass spectrometry.

To begin to characterize the antigens recognized by the scFvs, the molecular mass of the antigens is first assessed by immunoblot of protein extracts from the MDA-MB-231 or BT474 cells. The scFv will be diluted into 5% milk protein in PBS, incubated with the blocked PVDF membrane at 4° C. for 4 h, washed with PBS, incubated with a polyclonal anti-His tag antibody with secondary immunoenzymatic detection. Alternatively, if the antibody does not work for immunoblotting, the mass of the antigen can be assessed by immunoprecipitation. Immunoprecipitation would be conducted similar to the methods described in Singh et al. 2004, where the surface proteins would first be labeled by biotinylation, then immunoprecipitated and detected using streptavidin-HRP and a chemiluminescent substrate. Immunoprecipitation will be conducted by incubating the protein extracts with the scFv overnight at 4 degrees Celsius, followed by incubation with protein A-coupled agarose, as described in Singh et al. 2004.

To identify antigens immuno-affinity isolation (immunoprecipitation) is performed followed by mass spectrometry for peptide identification. Mass spectrometry requires as little as 50 femtomoles of protein contained in a band on an SDS-polyacrylamide gel (Griffin et al., 2001). Proteins isolated will be separated by gel electrophoresis and visualized by silver staining. Protein bands of the correct mass will be cut from the gel and submitted for analysis and identification. Peptide sequences obtained may be submitted for BLAST analysis and protein identification, using the publicly available Internet resources.

Determining the binding antigen's prevalence in normal and cancerous tissues. As one method to select for antibodies that bind preferentially to human breast cancer cells, the relative binding of isolated phage to tissue sections of normal and cancerous human breast tissues is examined. Tissue sections from normal and cancerous human breast tissue and the sections of normal tissue (for example, from reduction mammoplasty) are prepared. Frozen sections are fixed using 2% paraformaldehyde for 10 minutes, washed with PBS and blocked with for 2 hours using 10% goat serum in PBS. Purified scFvs are used to immunostain tissue sections to characterize the prevalence of their respective binding antigen in normal and cancerous tissues in vivo. The scFvs are diluted in 10% goat serum in PBS, incubated with the fixed sections at 4° C. for 4 h, washed with PBS, incubated with the biotinylated anti-Hs-tag antibody and secondary antibody-enzyme conjugate. The sections are counter stained with hematoxylin and examined for the presence of scFv reactivity.

Although the identification of disease-specific antigens is a result, the isolated antibodies may not exclusively bind to cancer cells. However, it is often valuable to have ligands that are targeted to antigens that are merely overexpressed on a diseased tissue. For example, HER2/neu is over-expressed on certain aggressive cancers, but is not uniquely expressed on these cancers. It is also found in cardiac muscle tissue, allowing for a potential site of toxicity for therapies targeted to this antigen. However, HER2-targeted immunoliposomes have demonstrated a threshold effect for receptor-mediated internalization, and thus activity, whereby at low antigen densities such as found in MCF7 breast cancer cells (<10,000 receptors/cell) internalization is essentially the same as nontargeted liposomes, and cells expressing moderate-to-high levels of the antigen (>100,000 receptors/cell) demonstrate rapid internalization and specific activity. This suggests that higher expression and not necessarily specific expression can be adequate for significant disease inhibiting activity.

Constructing scFv-targeted immunoliposomes and characterize their cancer cell targeting properties in vitro. Constructing liposomes and immunoliposomes. scFv-targeted immunoliposomes are constructed with either fluorescent dyes or for stably encapsulated anticancer drugs (such as doxorubicin, vinorelbine, or topotecan) and with antibody fragments conjugated specifically to the extraliposomal membrane using an activated Maleimide-PEG-distearoylphosphatidylethanolamine lipid anchor. The liposomes will be purified to remove any unencapsulated dye/drug and unconjugated protein, and will then be characterized for drug encapsulation efficiency, degree of scFv conjugation, and particle size. These liposomes will be used in subsequent experiments looking at in vitro binding, internalization, and targeted cytotoxicity of the targeted immunoliposomal drug and in vivo studies on the pharmacokinetics, acute toxicity, and antitumor efficacy of the various constructs.

Liposomes used in both in vitro and in vivo characterization of the scFv targeting properties can be prepared by any means known in the art. In particular, the liposomes may be prepared as follows: (1) the lipids; distearoylphosphatidylcholine (DSPC), cholesterol (Chol), and PEG-distearoylphosphatidylethanolamine (PEG-DSPE) are combined in a 3:2:0.3 molar ratio in a chloroform:methanol (9:1, vol:vol) solution and dried by rotary evaporation and under vacuum. (2) Fluorescent liposomes will be prepared by injection of an ethanolic solution of the lipids into an aqueous solution containing the fluorescent dye (35 mM pyranine) or for microscopy studies the lipophilic dye DiIC₁₈(3)-DS was included with the other lipids during liposome formation, followed by sizing using extrusion through polycarbonate filters with average pore sizes of 0.1 μm. (3) Unencapsulated fluorescent dye will be removed by Sephadex G-75 size exclusion chromatography, eluting with Hepes buffered saline (pH 6.5). (4) The resulting purified liposomes will be characterized with respect to size using photon correlation spectroscopy on a Coulter N4 plus particle size analyzer and for phospholipid content using by simple phosphate analysis.

Drug-loaded liposomes are prepared, for example, using transmembrane ammonium gradient according to the method of Haran et al. (1985). The unencapsulated ammonium salt, for example, 0.25 M ammonium sulfate, is removed by gel chromatography. Drug is added to the liposomes in the required ratios, typically 150 g Dox/mol phospholipid (PL) for doxorubicin and 350 g drug/mol PL for vinorelbine (VRL) and topotecan (TPT). The solution is then adjusted to a pH of 6.0-6.5 with 1 M NaOH and then incubated at 60° C. for 30 min to initiate loading. The reaction mixture is quenched on ice for 15 min and unloaded drug will be removed by Sephadex G-75 gel filtration chromatography and drug will be quantified by absorbance (498 nm for Dox, 277 nm for VRL, and 350 nm for TPT) in acidic isopropanol or acidic methanol and phospholipid by phosphate analysis. The drug loading efficiency is preferentially a minimum of 95% for optimal use.

Ligand-drug or cytotoxin conjugates can be prepared by first conjugating the ligand, such as antibody or scFv to a terminally-activated lipopolymer, such as, Maleimide-PEG-DSPE as described in U.S. Pat. No. 6,210,707 and by Nellis, et al., 2005a,b. Briefly, ScFv antibody fragments are cloned into an expression vector that allows for inclusion of a c-terminal cysteine and possibly purification via Ni2+−NTA chromatography. The protein is expressed in E. coli and purified using a combination of Ni2+−NTA, protein A sepharose, and ion-exchange chromatographies. ScFv dimers are reduced at the terminal disulfides using any suitable sulfhydryl reduction protocol, for example, by incubation with 10-20 mM mercaptoethylamine in a deoxygenated solution at pH 6.5, followed by purification by gel filtration chromatography using a Sephadex G-25 column to remove the mercaptoethylamine. The reduced scFv monomers will be conjugated to maleimide-PEG-distearoylphosphatidylethanolamine (Mal-PEG-DSPE) and inserted into fluorescent probe-(pyranine, ADS645-WS) or cytotoxic drug-(doxorubicin, vinorelbine, topotecan) loaded liposomes. In one embodiment, the ligand, such as antibody fragments employed herein, contain a single C-terminal cysteine engineered into the sequence to allow for specific chemical coupling. Typically, the resulting micellar conjugates are incubated with the liposomal drugs at 50-65° C. for 10-60 min, or at 30-40° C. overnight, followed by quenching on ice.

Determine cell binding, internalization, and in vitro cytotoxicity. Anti-PTMA immunoliposomes are compared to nontargeted liposomes, among immunoliposomes constructed with different antibodies, and at different antibody-to-liposome ratios to determine the relative degree of specificity for cell association and cell internalization and the optimum immunoliposome composition for high degrees of cell association and rates of internalization. Because the antibodies are added to the liposomes in what amounts to a multidisplay arrangement, for various particular applications it is important to determine what the optimum scFv-to-liposome ratio is for each antibody, as this may differ depending on the nature of the recognized epitope and the relative affinity of the antibody for its epitope.

To determine the extent of antigen-specific cellular uptake of anti-PTMA immunoliposomes, the cells that express the post-translationally modified antigen, as well as control cell lines that do not over express the target antigen, are grown in adherent state and incubated with immunoliposomes having various scFv density (5-100 scFv/liposome) at 37° C. typically for 4 hours and at a concentration of 50 μM PL. The cells extensively washed with phosphate-buffered saline are lysed within a detergent, and the liposomes are quantified by fluorometry of the liposome-encapsulated fluorescent marker. The amount of cell associated liposomal phospholipid is determined from concurrently analyzed liposome standards, and plotted as a function of antibody density to determine the optimum immunoliposome construct to move forward with in future studies. In many instances, the density of 5-50, typically 10-40 scFv/liposome is used.

Each antibody at its optimum density may be tested for relative rate of binding and internalization in different target cells, for example, using pyranine method (Daleke et al., 1990; Kirpotin et al., 1997; Mamot et al., 2003). The pH-dependent fluorescent dye, pyranine (also known as HPTS) has been previously used to report on the physical environment of the liposomes that entrap it, and thus on the rate of endocytosis. As the liposomes become endocytosed they appear in acidic compartments such as endosomes or lysosomes and can be quantitated with respect to the percent of cells internalized and percent of cells bound at the cell surface by measuring the fluorescence of pyranine at 512 nm upon excitation at both 413 nm (pH-independent isobestic point) and 454 nm (pH-dependent excitation). Liposomes and various immunoliposome constructs (50 μM PL) containing pyranine are added to cells (initially BT-474 or MDA-MB-231) plated at a density of 500,000 cells/well in six well plates and allowed to incubate for 1 h at 4° C., or for 5 min, 15 min, 30 min, 1 h, 2 h, 4 h, and 8 h at 37° C. The cells are washed with phosphate-buffered saline twice and detached by incubation in PBS+2 mM EDTA. The fluorescence in the cells is read at 512 nm while exciting at both 454 and 413 nm and the % of internalized, bound, and total liposomes/cell will be calculated as described in Kirpotin et al. (1997). As different cell lines may bind and internalize differently targeted liposomes more efficiently, the assay is typically repeated using more than one breast carcinoma cell lines, including, for example, SKBR-3, MDA-MB-435, and T47D.

Immunoliposome-mediate cytotoxicity is usually determined using drug-loaded liposomes (containing either VRL, DOX, or TPT) and immunoliposomes in several cell lines that express and do not express the post-translationally modified antigen recognized by the selected scFvs. The following exemplary protocol is suitable, based on MTT tetrazolium assay (Carmichael et al., 1987). Cells are plated at a density of 5,000-12,000 cells/well in 96 well cell culture plates. The cells are allowed to adhere overnight and then the various drug formulations (different anti-PTMA ILs drugs, nt-Ls drugs, and free drugs) are incubated with the cells for 2-4 h at 37° C., washed with PBS, and incubated for an additional 48 h in the appropriate media. The media are removed and a 0.5 mg/ml solution of 3-(4,5-dimethylthiazol-2-yl)-2,5-diphenyltetrazolium bromide is added in the medium to each well. The plates are incubated for an additional 2 h, the media removed, the precipitated formazan product solubilized in 70% isopropanol −0.1 N HCl and quantified by absorbance at 570 nm in a microtiter plate reader.

The internalization may be conveniently determined using fluorescent DiIC18(3)-DS labeled liposomes incubated with cells for 1 h at 4° C., for 4 h at 37° C., and for 1 h at 37° C. followed by washing with PBS to remove unbound liposomes and incubation for an additional 3 h at 37° C. Regular or confocal fluorescent microscopy is used to detect the presence of liposomes inside the cells incubated under the endocytosis-permissive conditions (37° C.) A punctate fluorescence pattern in a perinuclear region suggests internalization to late endosomes or lysosomes.

Characterization of pharmacokinetics, biodistribution, toxicity, and antitumor efficacy of scFv-targeted anti-PTMA immunoliposomes in vivo using animal models of human tumors. Typically, the inventive immunoliposomes are compared to control (non-targeted) liposomes in vivo with regard to pharmacokinetics, biodistribution, and anti-tumor efficacy. For example, to determine if insertion of these specific scFv-PEG-DSPE conjugates have any effect on clearance from the general circulation, both liposomal lipid and drug is quantified in rat d circulation in a pharmacokinetic study. Targeted immunoliposomes are compared to non-targeted liposomes in a biodistribution study using a human breast cancer xenograft that maintains the exposed transformation-specific epitope to determine if targeting has any effect on the accumulation of liposomal drug in both tumor and healthy tissues.

Drug-loaded immunoliposomes are compared to non-targeted drug-loaded liposomes and free drug in an in vivo efficacy study using the same human tumor xenograft study. One suitable treatment schedule includes three weekly i.v. injections of the drug at 80% of the MTD for the liposomal drug.

Pharmacokinetics and liposomal drug release rates of anti-PTMA immunoliposomes. Anti-PTMA immunoliposomes with significant and specific in vitro cytotoxicity are further studied in vivo using, for example, rodent models to identify those the immunoliposomes that have the desired long circulating half life and in vivo drug retention allowing specific antibody mediated delivery to tumor cells. The following are suitable exemplary protocols.

Rats with indwelling central venous catheters are injected with a 0.2-0.3 ml bolus of ³H—CHE-labeled (CHE=cholesteryl hexadecyl ether) liposomes containing the drug being studied. Blood samples (0.2-0.3 ml) are drawn at various times post injection using heparin-treated syringe. The pharmacokinetics of immunotargeted constructs is compared to nontargeted constructs to insure the process used for insertion of the antibody-lipid conjugate does not adversely affect drug retention in the liposomes by destabilizing the liposomal membrane or transmembrane gradient. The concentration of drug is determined in the plasma using HPLC analysis with UV absorbance or fluorescence detection by methods known in the art.

The liposome lipid is quantitated by scintillation radioactivity counting of H—CHE using conventional methods. The liposome preparations with known drug and 3H-CHE-lipid concentration can be used as standards. Radioactivity standards contain equal amount of diluted rat plasma to account for quenching. The percent of drug remaining in the liposomes is calculated by dividing the drug/lipid ratio in the blood samples by the drug/lipid ratio of the injected liposomes (taken as 100%). The rate constant of drug escape from the liposomes is determined by fitting the data to an appropriate, e.g., monoexponential, kinetic model. Pharmacokinetic parameters including the blood elimination rate constant, volume of distribution (Vd), clearance (CL), the mean residence time in the circulation (MRT), and the area under the concentration versus time curve (AUC∞) are determined by any routine pharmacokinetics computational method.

Evaluation of acute toxicity and anti-tumor efficacy of anti-PTMA-immunoliposomes. Prior to clinical use, it is further advantageous to find the toxicity and antitumor efficacy of the inventive immunoliposomes in animal models. The toxicity can be expressed, for example as maximum tolerated dose (MTD) of the drug, that is, the maximum dose that does not cause death or terminal irreversible morbidity in a representative number of animals within specified time. MTD is determined and compared to the MTD of untargeted liposomal VRL, TPT, and DOX and free drug (unencapsulated VRL, TPT, and DOX). The antitumor efficacy of liposomal and immunoliposomal drugs is compared in two different human xenograft models that have been shown to have the tumor-specific post-translation modification. An acute toxicology study determines an appropriate dose for administration of the liposomal or immunoliposomal drugs for antitumor efficacy studies. Anti-tumor efficacy studies determine the degree of improvement in antitumor efficacy accomplished through immunotargeting. The combination of these two studies provides evidence of the degree of improvement in the therapeutic window upon liposome encapsulation and immunotargeting.

As described above, alterations in post-translational modifications at the surface of cancer cells include protein cleavage by metalloproteases and altered protein glycosylation. These variables are also illustrated by the recent analysis of protein modifications within the dystroglycan glycoprotein complex in carcinoma cells. Dystroglycan forms the core of a transmembrane protein complex that mediates cell-extracellular matrix (ECM) interactions in muscle, neuronal and epithelial cells. Multiple post-translational events that modify the composition and function of this receptor complex at the cell surface in carcinoma cells have been identified. These events include cleavage of the N-terminal globular domain by furin, metalloprotease induced shedding of the β-DG subunit from the cell surface, and metalloprotease-mediated cleavage of the β-DG subunit. They also include the variable processing of carbohydrates in carcinoma cells that leads to a loss of laminin-binding functions. All of these events are summarized in FIG. 6.

For example, metalloprotease cleavage events are responsible for the shedding of β-DG from the cell surface, and for direct cleavage of the extracellular domain of β-DG, which is detected by the reduction in the molecular mass of β-DG from 43 kD to 31 kD. Both the shedding of β-DG, and the cleavage of β-DG are blocked by the incubation of the cells with the broad-spectrum MMP inhibitors GM6001 or BB2516. Detection of the cleaved form of p-DG can be completely eliminated after just 24 hours exposure to the MMP inhibitor.

New protein epitopes are exposed on the cell surface following both the shedding of α-DG and the cleavage of β-DG. These new binding sites include the newly exposed portions of the β-DG molecules, either from release of the associated α-DG or from cleavage of β-DG itself. DG cleavage and/or shedding can also expose new antigens on DG-associated molecules.

In addition to the regulation of the DG complex described herein, an aberrant form of alpha-DG is displayed on the surface of many carcinoma cells result of hypo-glycosylation of the alpha-DG subunit. The first evidence of this defect was obtained when antibodies directed at carbohydrate-dependent epitopes of a-dystroglycan failed to detect the molecule in a large percentage of carcinoma cell lines (see FIG. 7 right lanes. Subsequently, immunoprecipitation analysis of the surface-labeled dystroglycan complex, revealed the presence of the α-DG at the surface of these cells, but migrating at a lower molecular mass as the result of hypoglycosylation (FIG. 8a). The hypoglycosylation of this subunit also abolishes binding to the receptor ligand, laminin-1 (FIG. 8b). This hypoglycosylated form is predominant in advanced (invasive) cancer cell models and the loss of receptor function caused by this alteration is likely to contribute to the invasive behavior of cancer cells.

The exact origin of the defect in glycosylation is not yet certain, but may result from loss of glycosyltransferase activity, as already evident in some muscular dystrophies, or from altered regulatory pathways controlling post-translational protein modifications.

Attempts to detect DG in human cancers have revealed a loss of detection in breast, colon and prostate carcinoma cells. As observed in FIGS. 7 and 8, this loss of detection reflects to the absence of the functional dystroglycan isoform, and this loss correlates with progression of the disease. From these data one can conclude that hypoglycosylation of DG is likely to be unique to, or highly elevated in, cancers cells (particularly in metastatic cells).

In one embodiment, the cells carrying PTMA for phage library selection are mammary epithelial cells from transgenic mice in which this receptor's functions can be selectively deleted in cultured cells and in vivo via Cre-Lox recombination. These cells were be generated using the “floxed-DG” transgenic mouse line. In these mice, two “LoxP” DNA sequences have been inserted into non-coding regions of the DG gene (surrounding exon 2) without interfering with gene expression or function, yet the two LoxP sites flank DNA sequences that are critical for gene expression. Transient expression of the Cre recombinase in these normal cells induces recombination between the LoxP sites, disposing of intervening DNA sequences and specifically eliminating expression of the targeted gene. Therefore, introduction of the Cre transgene into mammary epithelial cells from homozygous “floxed-DG” animals permits the deletion of both copies of the DG gene and permits subsequent comparison of DG+/+ and DG−/− cell behavior.

To facilitate studies in cell culture, immortalized mammary epithelial cell lines have been established from the primary cultures of the floxed-DG transgenic mouse. Deletion of the DG gene has been accomplished by transient expression of the Cre transgene through adenoviral infection of the “floxed-DG” primary cultures and cell lines, and established completely DG−/− cell populations from each of the immortalized cell lines (through limiting dilution colony selection). The DG cDNAs were then stably re-expressed in the completely DG−/− cell populations by infection with the pBM-IRES/puro retroviral vector, encoding the human DG cDNA and the puromycin resistance gene. This vector produces a polycystronic message encoding the transgene and antibiotic resistance, assuring co-expression of the two. In addition to the wild-type (WT) DG cDNA, mutants of DG have been created and analyzed, including: 1) a deletion of the cytoplasmic domain (D1); 2) a mutation of the cleavage site separating the α and β-DG subunits, producing DG as a single high molecular weight molecule, rather than two separate subunits (labeled “MC”); and 3) a mutation within the N-terminal globular domain of DG (labeled “HG” for hypoglycosylation), which was shown to disrupt proper DG glycosylation. Cells were infected with each of these viruses, and the infected cell populations selected in puromycin-containing medium. A control cell population, lacking DG expression, was established by infecting cells with the viral vector alone (labeled “V”). Immunoblots of total protein extracts from infected cell populations revealed the expected molecular masses of each mutant molecule. They revealed the absence of α and β-DG in the control (V) population, and the correct molecular mass of the WT DG subunits. They revealed the smaller DG subunit in the D1 mutant, which retained normal DG expression. They also revealed the absence of the DG subunit in the higher mass MC mutant, because this DG is not cleaved into two subunits (FIG. 8A). The HG mutation created a normal β-DG subunit, but the α-DG subunit was not properly glycosylated and, therefore, not detected by the IIH6 monoclonal antibody. This result is consistent with the known requirements for α-DG modification by the glycosyl-transferase LARGE.

The functions of cells re-expressing “wild-type” (WT) or mutant DG cDNAs were then assayed. In assays of laminin assembly, using fluorescein-labeled laminin, the vector population showed no detectable laminin binding, whereas the WT population showed a restoration of laminin binding and assembly (FIG. 8B). Expression of the D1 and MC mutants also restored laminin assembly, showing that the membrane-proximal region of the DG cytoplasmic domain is not essential for this DG function, nor is the cleavage of DG into two subunits. As predicted, the HG mutant did not efficiently assembly laminin, as the result of improper glycosylation, which disrupts laminin binding. Thus, the cell populations that completely lack DG expression, and a derived cell population that expresses the WT and hypoglycosylated form of the human α-DG were produced. These customized cell populations have unique and surprising advantages in the screening of the phage display antibody library for the isolation of antibodies binding distinct post-translational modifications of dystroglycan.

Acute toxicities of free drug, liposome-encapsulated drug, and immunoliposomal drug are compared by determining the maximum tolerated dose (MTD) following single i.v. injection in regular (immunocompetent) mice (e.g., female Swiss Webster mice). Toxicological and efficacy studies in animal models are well known in the art. (See, for example, H. H. Fiebig and A. M. Burger, editors, Contributions to Oncology, vol. 54, Karger, N.Y., 1999). For example, one suitable toxicological protocol includes administration to the animals, in the groups of two, of the increasing doses of the liposomes (with the dose escalation factor 1.8) until at least one animal experiences unacceptable morbidity; continuing the same process from the next lowest does with the dose escalation factor 1.15, and confirming the non-toxic dose in the group of 5 animals.

The exemplary protocol for antitumor efficacy studies for anti-PTMA immunoliposomes includes subcutaneous model of human breast carcinomas (MDA-MB468, BT474, or other, depending on the antigen to be targeted) in nude mice. The tumor cells are propagated in culture and inoculated into flank area of NCR nu/nu homozygous athymic female nude mice, typically at 10⁷ cells/injection. When the animals develop tumors in the range of 100 mm³-300 mm³ the mice are randomly assigned to six treatment groups of 10-12 animals/group, and treated with three weekly i.v. injections of the following agents: 1) Control (HEPES-buffered saline pH 6.5); 2) Free drug (either Dox, TPT, or VRL) 3) Ls-drug; 4) ILs-drug (Ab #1); 5) ILs-drug (Ab #2); 6) ILs-drug (control Ab). The dose per injection is determined on the basis of acute toxicity studies above, and typically will be taken as ⅔ of the acute MTD. The animal weight and tumor size are monitored twice weekly as described above. The weight of tumor, when appropriate, is subtracted from the animal weighing results to obtain animal body weight. The animals are observed for at least 60 days following tumor inoculation. Animals with tumors reaching 20% of the body weight, or those with signs of tumor necrosis, ulceration, or general morbidity, are euthanized for humane reasons. If any of the animals show complete regressions of the tumor, the tumor site is preserved for pathological examination for residual microscopic disease. A second study is performed similarly except using a second drug as the encapsulated chemotherapeutic. The drug chosen for these antitumor efficacy studies is determined based on targeted activity in breast cancer cells, the results of acute toxicology studies (i.e. the benefit of liposomal and immunoliposomal encapsulation, if any), and in vivo formulation stability and pharmacokinetics.

At least one of these new immunoliposomal drugs is examined in a second efficacy study to determine the dose dependency of the response. In a similar experimental settings, the tumor growth among the treatment groups receiving free, non-targeted liposomal, or immunoliposomal drug at 20%, 40%, or 80% of MTD is determined in at least one target-competent tumor model.

The effect of anti-PTMA targeting on the immunoliposome antitumor efficacy is further tested in a control study using the tumors that does not display the post-translational modification targeted by the selected antibodies. The lack of immunoliposome uptake is confirmed in cell uptake studies prior to initiation of the study. In this study, the chosen human breast cancer cell line are grown in a xenograft model in NCR nu/nu female mice similar to that described above. At least the following six groups will be compared: 1) Control (HEPES-buffered saline pH 6.5); 2) Ls-drug at 80% of MTD, 3) ILs-drug (using best antibody from above studies at 80% of MTD), 4) 2nd antibody from above studies at 80% of MTD. The tumor growth data are statistically compared across the treatment groups to determine if the presence of the developed antibody ligands has effect on the liposomal drug efficacy in the absence of the target antigen on tumor cells. This study further addresses the antigen specificity of the developed targeted drug carriers in vivo.

The above examples are presented to illustrate, but not to limit, the invention. A skilled artisan would recognize many ways in which the invention can be practiced without departure from the meaning and scope of the presented disclosure and the claims.

1. A composition comprising a pharmaceutical entity linked to a ligand wherein the ligand specifically binds to a post-translationally modified antigen (PTMA).

2. The composition of claim 1, wherein the pharmaceutical entity is selected from the group consisting of a cytotoxin, a pharmaceutical compound, a drug carrier, a nanoparticle, a polynucleotide, a detectable marker, and a liposome.

3. The composition of claim 1, wherein the PTMA is cancer cell-specific.

4. The composition of claim 1, wherein the ligand is selected from the group consisting of a polypeptide, a nucleic acid, an aptamer, a protein, a polysaccharide, an antibody, an antibody fragment, a Fab′ fragment, an Fv antibody fragment, a single domain antibody, a single-chain antibody, and a single-chain Fv antibody.

5. The composition of claim 3, wherein the PTMA is selected from the group consisting of a glycoprotein, a lipoprotein, a protein comprising a transmembrane domain, a polysaccharide, a cell surface antigen, and a cell surface receptor.

6. The composition of claim 1, wherein the PTMA comprises a cell surface protein post-translationally modified by proteolysis in a cancer cell phenotype.

7. The composition of claim 6, wherein the PTMA comprises a cell surface protein post-translationally modified by a metalloproteinase.

8. The composition of claim 1, wherein the PTMA comprises dystroglycan.

9. The composition of claim 2, wherein the liposome comprises a drug.

10. The composition of claim 9, wherein the drug is an anticancer drug.

11. The composition of claim 10, wherein the anticancer drug is selected from the group consisting of an anthracycline, a vinca alkaloid, and a camptothecin derivative.

12. The composition of claim 10, wherein the drug is selected from the group consisting of doxorubicin, vinorelbine, irinotecan, and topotecan.

13. The composition of claim 2, wherein the ligand is linked to the liposome via a hydrophilic polymer spacer.

14. The composition of claim 13, wherein the hydrophilic polymer spacer comprises poly(ethylene glycol).

15. The composition of claim 1, wherein the PTMA antigen comprises a cell surface PTMA.

16. The composition of claim 15, wherein the ligand, when bound to the cell-surface PTMA, internalizes into the cell.

17. A method of identifying a ligand that interacts with a post-translationally modified antigen (PTMA) comprising:
(a) contacting a ligand library having a plurality of members with cells that display the PTMA, and
(b) separating the cells from the library members that do not associate with the cells.

18. A method of identifying a ligand that interacts with a post-translationally modified antigen (PTMA) comprising:
(a) contacting a ligand library having a plurality of members with cells of a first cell line that displays a precursor antigen;
(b) separating the ligand library members that do not associate with the cells of the first cell line;
(c) contacting the ligand display library members separated in (b) with the cells of the second cell line wherein the second cell line is obtained by a process comprising inducing the first cell line to modify the precursor antigen into the post-translationally modified antigen.

19. The method of claim 18, wherein the second cell line is obtained by contacting the first cell line with a growth factor, an enzyme, a chemical factor, or a physical stimulus.

20. The method of claim 19, wherein the growth factor is a transforming growth factor.

21. The method of claim 19, wherein the enzyme is selected from the group consisting of a protease, a glycosidase, a kinase, a phosphatase, a sulfatase, a lipid transferase, a farnesyltransferase, a myristoyl transferease, and a palmitoyl transferase.

22. The method of claim 19, wherein the physical stimulus is a change in temperature, acidity, redox potential, oxygen content, light or ionizing radiation.

23. The method of claim 17 wherein the ligand library is a ligand display library.

24. The method of claim 18 wherein the ligand library is a ligand display library.

25. A ligand identified by the method of claim 17 or claim 18.

26. A PTMA that specifically binds to a ligand identified by claim 17 or claim 18.

27. A ligand that binds to a post-translationally modified antigen (PTMA) expressed on the outer surface of a cell, and further internalizes into said cell.

28. A pharmaceutical composition comprising a ligand of claim 25.

29. A diagnostic composition comprising a ligand of claim 25 linked to a detectable marker.

30. A composition comprising a PTMA of claim 26.

31. The composition of claim 30, wherein the PTMA is a dystroglycan polypeptide that comprises hypoglycosylation.

32. The composition of claim 30, comprising an adjuvant.

33. A method of treatment or diagnosis of a disease in a subject comprising administering to the subject an effective amount of a composition of any of the claims 25-26 or 27.

34. The method of claim 33, wherein the disease is cancer.

35. The method of claim 34, wherein the disease is carcinoma.

36. The method of claim 35, wherein the disease is the cancer of a mammary gland.

37. A method for selecting a ligand that specifically internalizes into a cell bearing a cellular marker derived from a precursor antigen, the method comprising the steps of claim 17, wherein the contacting of step (a) is under conditions allowing for internalization of the ligand library members into the cell, and further includes the step of removing the members of ligand display library that are external to the cell.

38. A method for selecting a ligand that specifically internalizes into a cell bearing a cellular marker derived from a precursor antigen, the method comprising the steps of claim 18, wherein the contacting of at least step (c) is under conditions allowing for internalization of the ligand library members into the cell, and further includes the step of removing the members of ligand display library that are external to the cell.

39. The method of claim 17, wherein the ligand is a peptide, an aptamer, an antibody, an immunoglobulin, a single chain antibody, an antibody fragment, a Fab antibody fragment, a Fab′ antibody fragment, a single domain antibody, an Fv antibody fragment, or a single chain Fv antibody fragment.

40. The method of claim 17, wherein the library is a phage display library.

41. The method of claim 19, wherein the modification comprises activating or inhibiting the activity of an enzyme.

42. The method of claim 18, wherein the modifying of the first cell line comprises contacting the first cell line with an inhibitor of a proteolytic enzyme, an activator of a proteolytic enzyme, or a growth factor.

43. The method of claim 18, wherein the ligand is dystroglycan, modifying is by a metalloprotease and/or TGF-beta.

44. The method of claim 18, wherein the ligand is alpha-dystroglycan.

45. A method of eliciting an immune response in a subject comprising administering a composition of any one of claim 30-32.

46. A vaccine comprising the composition of any one of claims 30-32.
